(19) United States
(12) Patent Application Publication
(10) Pub. No.: US 2014/0341822 A1
(43) Pub. Date: Nov. 20, 2014

(54) Associative Thickeners Based on Hyperbranched Polymers

(71) Applicants: Holger Türk, Mannheim (DE); Volker Wendel, Seeheim-Jugenheim (DE); Monika Haberecht, Ludwigshafen (DE); Daniel Stadler, Shanghai (CN); Bernd Bruchmann, Freinsheim (DE)

(72) Inventors: Holger Türk, Mannheim (DE); Volker Wendel, Seeheim-Jugenheim (DE); Monika Haberecht, Ludwigshafen (DE); Daniel Stadler, Shanghai (CN); Bernd Bruchmann, Freinsheim (DE)

(73) Assignee: BASF SE, Ludwigshafen (DE)

(21) Appl. No.: 14/237,265

(22) PCT Filed: Jul. 26, 2012
(86) PCT No.: PCT/EP2012/064687

§ 371 Date: Aug. 11, 2014

Related U.S. Application Data
(60) Provisional application No. 61/515,333, filed on Aug. 5, 2011.

(30) Foreign Application Priority Data
Aug. 5, 2011 (EP) 11176673.9

(51) Int. Cl. A61K 8/87; C08G 18/44; C08G 18/64; A61Q 9/04; A61Q 17/04; A61Q 15/00; A61Q 5/12; C08G 18/48; C08G 18/50

(52) U.S. Cl. CPC A61K 8/87; C08G 18/4833; C08G 18/44; C08G 18/6415; C08G 18/5024; A61Q 17/04; A61Q 15/00; A61Q 5/12; A61Q 9/04; C08G 2105/02; A61K 2800/48; A61K 2800/10
USPC 424/59; 525/452; 514/772.1; 424/65; 424/70.11; 8/161

(57) ABSTRACT

The present invention relates to associative polyurethane thickeners which comprise hyperbranched polymers in polymerized-in form, to the preparation of these thickeners, and to the use thereof, particularly in cosmetic preparations.

The present invention relates to associative polymeric thickeners which comprise hyperbranched polymers in polymerized-in form, to the preparation of these thickeners, and to the use thereof as thickeners for aqueous preparations, particularly for aqueous, cosmetic preparations.

Associative thickeners based on polyurethane form part of the prior art. Polyurethane solutions or dispersions in water-thinnable aqueous or predominantly aqueous phase are referred to by the person skilled in the art as HEUR thickeners. They are described in detail, for example, in U.S. Pat. No. 4,079,028 and U.S. Pat. No. 4,155,892.

The “stellate products” (group B) and “complex polymers” (group C) described in U.S. Pat. No. 4,079,028 (Rohm & Haas) comprise polyurethanes into which polyhydric alcohols have been polymerized. The polyhydric alcohols are low molecular weight compounds such as, for example, trimethylolpropane, pentaerythritol, sorbitol, erythritol, sorbitol, mannitol or dipentaerythritol.

EP 1566393 (Cognis) describes thickeners based on an aqueous preparation of nonionic, water-dispersible or water-soluble, polyurethanes which can be prepared by reacting (a) one or more polyfunctional isocyanates with (b) one or more polyetherpolyols, (c) one or more monofunctional alcohols and (d) if desired one or more polyfunctional alcohols, where the compounds (d) comprise no further functional groups apart from the OH groups. The polyfunctional alcohols (d) comprise at least predominantly trifunctional alcohols, such as, for example, glycerol or preferably trimethylolpropane.

EP 1584331 A1 (Shiseido) describes polyurethane thickeners for cosmetic preparations, where the polyurethanes can also be branched. The underlying polyols and the alkoxylated derivatives thereof are described in sections [38] and [39].

EP 725097 A1 (Bayer) likewise describes thickeners based on polyurethanes. Branches can optionally be introduced into the polyurethanes by virtue of the component a4). Component a4) are 3- to 6-hydric alcohols in the molecular weight range 92 to 600, preferably 92 to 400 and particularly preferably 92 to 200, such as, for example, glycerol, trimethylolpropane, pentaerythritol and/or sorbitol.

EP 978522 (National Starch) describes branched polyurethane thickeners of the following formula

(Y₁Z)ₙ-A-(ZY₂X′)ₘ

In this, A is a hydrophilic polyol and is preferably selected from trimethylolpropane, [2-ethyl-2-(hydroxymethyl)-1,3-propanediol], pentaerythritol, glycerol and sorbitol.

U.S. Pat. No. 4,327,008 (PPG Industries) describes polyurethane thickeners with a branched structure, urea bonds and hydrophobic, terminal groups, and also the use thereof in coatings. The polymers comprise, as building blocks, polyfunctional compounds such as polyfunctional alcohols or amines, which can be alkoxylated.

EP 307775 (Rheox) describes polyurethane thickeners with a branched basic structure. The branches are introduced via a modifying agent, which is reacted with the polyisocyanate, the polyetherdiol and the monofunctional hydrophobic radical. The branching agent likewise comprises a hydrophobic radical and additionally at least two functional groups that are reactive toward isocyanate.

US 2009/0082483 A1 describes polyurethane foams based on the reaction products of polyisocyanates and polyglycerol which is hydrophobically modified prior to the urethanization by transesterification with naturally occurring polyol esters.

WO 2009/135857 discloses polyurethanes as rheology modifiers, in particular as thickeners for cosmetic preparations. The polyurethanes disclosed do not comprise polymerized-in hyperbranched polymers.

WO 2010/130599, WO 2007/125028 and WO 2006/087227 disclose polymers comprising polymerized-in, hyperbranched polymers. The polymers also comprise alkyl radicals which are derived from polymerized-in alcohols. These are, however, short-chain alkyl radicals, in particular methyl radicals.

Hyperbranched or dendrimeric polyurethanes are known from the literature. For the synthesis of such hyperbranched polyurethanes, preference is given to using ABₓ monomers which have both isocyanate groups and also groups which can react with isocyanate groups to form a linkage. x is a natural number between 2 and 8. Preferably, x is 2 or 3. Either A is the isocyanate groups and B is groups that are reactive with these, or vice versa. This substance class has hitherto not been described as thickeners for aqueous systems.

The groups reactive with the isocyanate groups are preferably OH groups, meaning that urethane bonds are formed.

The ABₓ monomers can be prepared in a known manner by means of various techniques.

ABₓ monomers can be synthesized for example by the method disclosed by WO 97/02304 using protective group techniques. One example is the technique of producing a AB₂ monomer from 2,4-tolylene diisocyanate (TDI) and trimethylolpropane, where firstly one of the isocyanate groups of the TDI is capped in a known manner, for example by reaction with an oxime. The remaining free NCO group is reacted with trimethylolpropane, where one of the three OH groups reacts with the isocyanate group. After cleaving off the protective group, a molecule with one isocyanate group and 2 OH groups is obtained.

The ABₓ molecules can be synthesized particularly advantageously in accordance with the method disclosed by DE-A 199 04 444, in which no protective groups are required. In this method, di- or polyisocyanates are used and reacted with compounds which have at least two groups that are reactive with isocyanate groups. At least one of the reactants has groups with a different reactivity compared to the other reactants. Preferably, both reactants have groups with a different reactivity compared with the other reactants. The reaction conditions are selected such that only certain reactive groups can react with one another.

The present invention had as its object to provide thickeners suitable for cosmetic applications which, compared to the known thickeners, are characterized by the fact that higher viscosity values can be attained than with conventional associative thickeners.

This object was achieved by the thickeners, also called P, MP1 or MP2 below, which are the subject of the present invention and which are described in more detail below.

These thickeners according to the invention have numerous advantages compared with thickeners known from the prior art. They are distinguished, inter alia, by an increase in water solubility, by the adaptability of the molecular structure (tailoring) to different requirements, by improved cosmetic properties such as, for example, a more effective skin moisturization, by an increase in the bioavailability and the solubility of active ingredients and effect substances such as e.g. photoprotective agents, by an increased accumulation and/or adhesion to the skin, by an improved compatibility with further constituents of cosmetic preparations and consequently, for example, increase in the stability of emulsions.

In particular, the thickeners according to the invention have the advantage of providing stable thickened compositions in the temperature range from about 35 to about 40° C., whereas thickeners known from the prior art no longer do this in this temperature range. This is of particular importance when using the thickeners in cosmetic formulations which are to be used in countries having high outside temperatures.

Furthermore, the thickeners according to the invention have the advantage that they are thickeners based on polyurethane which, compared with the conventional polyurethane thickening compositions, for a comparatively lower intrinsic viscosity of the thickening compositions in their formulation form, bring about an increased viscosity of the thickened product for the same use amount.

The present invention provides polymers P comprising, in polymerized-in form,

a) at least one polyisocyanate
b) at least one alcohol of the general formula I

R¹O—R²ₙOH (I)

where
R¹ is selected from C₆-C₄₀-alkyl, C₆-C₄₀-alkenyl, C₃-C₁₀-cycloalkyl, C₆-C₃₀-aryl and C₇-C₄₀-arylalkyl,
R² is selected from C₂-C₁₀-alkylene, C₆-C₁₀-arylene and C₇-C₁₀-arylalkylene,
n is selected from 0 to 200,
c) at least one hyperbranched polymer HB with functional groups, where, for the average number f of functional groups per molecule of the hyperbranched polymer, 3<f<100, in particular 3<f<20 applies,
with the proviso that the hyperbranched polymer is not selected from hyperbranched polyetherpolyols,
d) optionally at least one compound different from b) and c) and having a molecular weight of at least 300 g/mol comprising
i. at least two OH groups and
ii. at least two groups selected from ether groups and ester groups,
   e) optionally further compounds different from b) to d) and having 1 to 10 groups that are reactive toward isocyanate groups per molecule.

In a preferred embodiment, the polymers according to the invention are water-soluble or water-dispersible.

Within the context of this invention, “water-soluble” means that at least one gram, preferably at least 10 grams, of the substance referred to as water-soluble, thus for example of the polymers according to the invention, are soluble in 1 liter of demineralized water to give a solution that is clear to the human eye.

Within the context of this invention, “water-dispersible” means that at least one gram, preferably at least 10 grams, of the substance referred to as water-dispersible, thus for example of the polymers according to the invention, are dispersible in 1 liter of demineralized water without sediment with a maximum average particle size of 1 μm.

In a preferred embodiment, the polymers according to the invention are uncrosslinked. Within the context of this invention, “uncrosslinked” means that a degree of crosslinking of less than 15% by weight, preferably of less than 10% by weight, and in particular less than 5% by weight, determined via the insoluble fraction of the polymers, is present. The insoluble fraction of the polymers is determined by extraction for 4 hours with the same solvent as is used for the gel permeation chromatography for determining the molecular weight distribution of the polymers, i.e. tetrahydrofuran, dimethylacetamide or hexafluoroisopropanol, depending on in which solvent the polymers are more soluble, in a Soxhlet apparatus and, after drying the residue to constant weight, weighing the remaining residue.

### a) Polyisocyanate

According to the present invention, polyisocyanates are compounds with at least two isocyanate groups per molecule. Suitable polyisocyanates preferably comprise on average 2 (diisocyanates) to 4 NCO groups per molecule, with diisocyanates being particularly preferred.

By way of example, suitable isocyanates which may be mentioned are 1,5-naphthylene diisocyanate, 4,4′-diphenylmethane diisocyanate (MDI), hydrogenated MDI (H₁₂MDI), xylylene diisocyanate (XDI), tetramethylxylene diisocyanate (TMXDI), 4,4′-diphenyl-dimethylmethane diisocyanate, di- and tetraalkyldiphenylmethane diisocyanate, 4,4-dibenzyl diisocyanate, 1,3-phenylene diisocyanate, 1,4-phenylene diisocyanate, the isomers of tolylene diisocyanate (TDI), optionally in a mixture, 1-methyl-2,4-diisocyanatocyclohexane, 1,6-diisocyanato-2,2,4-trimethylhexane, 1,6-diisocyanato-2,4,4-trimethylhexane, 1-isocyanatomethyl-S-isocyanato-1-trimethylcyclohexane, 4,4′-diisocyanatophenylperfluoroethane, tetramethoxybutane 1,4-diisocyanate, butane 1,4-diisocyanate, hexane 1,6-diisocyanate (HDI), dicyclohexylmethane diisocyanate, cyclohexane 1,4-diisocyanate, ethylene diisocyanate, phthalic acid bisisocyanatoethyl ester, isophorone diisocyanate (IPDI).

In a preferred embodiment, the polymers P according to the invention comprise condensed-in cycloaliphatic or aliphatic diisocyanate radicals, particularly preferably aliphatic diisocyanate radicals.

Examples of suitable aliphatic diisocyanates a) which may be mentioned are: 1,4-butylene diisocyanate, 1,12-dodecamethylene diisocyanate, 1,10-decamethylene diisocyanate, 2-butyl-2-ethylpentamethylene diisocyanate, 2,4,4- or 2,2,4-trimethylhexamethylene diisocyanate and in particular hexamethylene diisocyanate (hexane 1,6-diisocyanate, HDI).

Examples of suitable cycloaliphatic diisocyanates a) which may be mentioned are: isophorone diisocyanate (IPDI), 2-isocyanatopropylcyclohexyl isocyanate, 4-methylcyclohexane 1,3-diisocyanate (H-T D I) and 1,3-bis(isocyanatomethyl)-cyclohexane. Also so-called H₁₂-MDI or diisocyanates termed “saturated MDI”, such as e.g. 4,4′-methylenebis(cyclohexyl isocyanate) (alternatively also called dicyclohexylmethane 4,4′-diisocyanate) or 2,4′-methylenebis(cyclohexyl) diisocyanate may be present as radicals in the polyurethanes according to the invention.

In a preferred embodiment, a) is or comprises hexamethylene diisocyanate. In a further preferred embodiment, a) is or comprises isophorone diisocyanate. Of course, mixtures of polyisocyanates can also be used as a).

### b) Alcohol of the General Formula I

The polymers P according to the invention comprise, in polymerized-in form, at least one alcohol of the general formula I

R¹O—R²ₙOH (I)

where R¹ is selected from C₆-C₄₀-alkyl, C₆-C₄₀-alkenyl, C₃-C₁₀-cycloalkyl, C₆-C₃₀-aryl, C₇-C₄₀-arylalkyl, R² is selected from C₂-C₁₀-alkylene, C₆-C₁₀-arylene, C₇-C₁₀-arylalkylene and n is selected from 0 to 200.

In one embodiment, R¹ is C₆-C₄₀-alkyl. In a preferred embodiment, R¹ is a C₆-C₃₀-alkyl radical, further preferably a C₈-C₂₆-alkyl radical, particularly preferably a C₁₂-C₂₆-alkyl radical and very particularly preferably a C₁₂-C₂₀-alkyl radical.

R¹ is selected, for example, from radicals of linear or branched alkanes such as hexane, heptane, octane, 2-ethylhexane, nonane, decane, undecane, dodecane, tridecane, isotridecane, tetradecane, pentadecane, hexadecane, heptadecane, octadecane, nonadecane, eicosane, heneicosane, docosane, tricosane, isotricosane, tetracosane, pentacosane, hexacosane, heptacosane, octacosane, nonacosane, triacontane, 2-octyldodecane, 2-dodecylhexadecane, 2-tetradecyloctadecane, 2-decyltetradecane, or monomethyl-branched isooctadecane.

In one embodiment, R¹ is selected from C₆-C₄₀-alkenyl. Suitable C₆-C₄₀-alkenyl radicals can be straight-chain or branched. Preference is given here to predominantly linear alkenyl radicals, as also occur in natural or synthetic fatty acids and fatty alcohols, and also oxo alcohols, which are mono-, di- or polyunsaturated. These include e.g. n-hexenyl, n-heptenyl, n-octenyl, n-nonenyl, n-decenyl, n-undecenyl, n-dodecenyl, n-tridecenyl, n-tetradecenyl, n-pentadecenyl, n-hexadecenyl, n-heptadecenyl, n-octadecenyl, n-nonadecenyl.

In one embodiment, R¹ is selected from C₃-C₁₀-cycloalkyl, where cycloalkyl is preferably cyclopentyl, cyclohexyl, cycloheptyl or cyclooctyl.

In one embodiment, R¹ is selected from C₆-C₃₀-aryl, where aryl comprises unsubstituted or substituted aryl groups and is preferably selected from phenyl, tolyl, xylyl, mesityl, naphthyl, fluorenyl, anthracenyl, phenanthrenyl, naphthacenyl and in particular from phenyl, tolyl, xylyl and mesityl.

In one embodiment, R¹ is selected from C₇-C₄₀-arylalkyl. Arylalkyl stands for groups which comprise both alkyl and aryl radicals, these arylalkyl groups being joined to the compound carrying them either via the aryl radical or via the alkyl radical. For example, R¹ can be selected from the arylalkyl radicals described in EP 0 761 780 A2, p. 4, I. 53-55.

In one embodiment, R¹ is a branched alkyl radical. Preferably, the side chains of such branched alkyl radicals are likewise alkyl radicals or alkylene radicals, particularly preferably alkyl radicals, in particular unbranched alkyl radicals.

In one embodiment, the side chains of the branched alkyl radicals R¹ have a chain length of at most 6, preferably of at most 4, carbon atoms.

In one embodiment, the branches are considerably shorter than the main chain. In one embodiment, each branch of R¹ has a chain length which corresponds at most to half of the chain length of the main chain of R¹. In one embodiment, the branches are considerably shorter than the main chain. In a preferred embodiment, the branched R¹ are iso- and/or neoalkyl radicals. In a preferred embodiment, the branched alkyl radicals R¹ used are radicals of isoalkanes. Particular preference is given to a C₁₃-alkyl radical, in particular an iso-C₁₃-alkyl radical.

In another embodiment, R¹ comprises branched alkyl radicals, the side chains of which have a chain length of at least 4, preferably of at least 6, carbon atoms.

In a preferred embodiment, R² in the general formula (I) is selected from —CH₂—CH₂—, —CH(CH₃)—CH₂— and mixtures thereof, particularly preferably —CH₂—CH₂—.

In a preferred embodiment, n is selected from the range 10 to 100.

In general, b) can also be a mixture of different alcohols.

In a preferred embodiment of the invention, at least one alcohol b) is selected from alkoxylated alcohols. Preferred alkoxylated alcohols are ethoxylated alcohols (R²=—CH₂—CH₂—), propoxylated alcohols (R²=—CH(CH₃)—CH₂—) and alcohols, which are either ethoxylated or propoxylated. In this connection, the ethylene oxide and propylene oxide units can be in random or blockwise distribution.

Suitable alcohols b) are, for example, the alkoxylated, preferably ethoxylated
linear alcohols from natural sources or from the Ziegler build-up reaction of ethylene in the presence of aluminum alkyl catalysts. Examples of suitable linear alcohols are linear C₆-C₃₀-alcohols, in particular C₁₂-C₃₀-alcohols. Particularly preferred alcohols which may be mentioned are: n-dodecanol, n-tetradecanol, n-hexadecanol, n-octadecanol, n-eicosanol, n-docosanol, n-tetracosanol, n-hexacosanol, n-octacosanol, and/or n-triacontanol, and also mixtures of the aforementioned alcohols, for example NAFOL® grades such as NAFOL® 22+(Sasol).
Oxo alcohols such as, for example, isoheptanol, isooctanol, isononanol, isodecanol, isoundecanol, isotridecanol (for example Exxal® grades 7, 8, 9, 10, 11, 13).
Alcohols which are branched in the 2 position; these are the Guerbet alcohols known to the person skilled in the art which are accessible by dimerization of primary alcohols via the so-called Guerbet reaction. Particularly preferred alcohols which may be mentioned here are: Isofol®12 (Sasol), Rilanit®G16 (Cognis).
Alcohols which are obtained by the Friedel-Crafts alkylation with oligomerized olefins and which then comprise an aromatic ring as well as a saturated hydrocarbon radical. Particularly preferred alcohols which may be mentioned here are: isooctylphenol and isononylphenol.
Alcohols of the general formula (4) of EP 761780 A2, p. 4

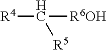

or alcohols of the general formula (5) of EP 761780 A2, p. 4

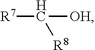

where
R⁴, R⁵, R⁷ and R⁸, independently of one another, have the meaning described in EP 761780 A2, p. 4, lines 45 to 58; preferably, R⁴, R⁵, R⁷ and R⁸, independently of one another, are alkyl radicals having at least 4 carbon atoms and the total number of carbon atoms of the alcohols is at most 30,
R⁶ is an alkylene radical such as, for example, —CH₂—, —CH₂—CH₂—, —CH₂—CH(CH₃)—; for example, mention may be made here of 2-decyl-1-tetradecanol as suitable alcohol.

In one embodiment, at least one alcohol b) is a mixture of ethoxylated linear C₁₆-C₁₈-fatty alcohols.

In one embodiment, at least one alcohol b) is a linear, nonionic compound of the structural formula RO(CH₂CH₂O)ₓH, where R is a linear C₁₆-C₁₈-alkyl radical, and x is selected from 3, 5, 7, 8, 11, 13, 18, 25 or 80, preferably x is selected from 11, 13, 18, 25 or 80. Such ethoxylated, linear fatty alcohols are commercially available for example as Lutensol® AT11 or Lutensol® AT80.

In one embodiment, at least one alcohol b) is selected from compounds of the structural formula RO(CH₂CH₂O)ₓH, where R is a linear C₈-C₃₀-alkyl radical, preferably linear C₁₆-C₁₈-alkyl radical, and x is selected from 4 to 30.

In a further embodiment, at least one alcohol b) is selected from compounds of the structural formula RO(CH₂CH₂O)ₓH, where R is a linear C₈-C₃₀-alkyl radical, preferably linear C₁₆-C₁₈-alkyl radical, and x is selected from 30 to 80.

In one embodiment of the invention, b) is selected from mixtures of ethoxylated linear and ethoxylated branched long-chain alcohols, in particular mixtures of the aforementioned types.

In a further embodiment, b) is selected from ethoxylated iso-C₁₃-oxo alcohols and mixtures thereof.

In one embodiment, at least one alcohol b) is a branched, nonionic compound of the structural formula RO(CH₂CH₂O)ₓH, where R is a C₁₃-alkyl radical, preferably an iso-C₁₃-alkyl radical, and where x=3, 5, 6, 6.5, 7, 8, 10, 12, 15 or 20, preferably x selected from 10, 12, 15 or 20 is used. Commercially, one such ethoxylated, alkyl-branched alcohol is available, for example as Lutensol® TO10.

In a further embodiment, b) is selected from mixtures consisting of or comprising ethoxylated C₁₆-C₁₈-fatty alcohols and ethoxylated iso-C₁₃-oxo alcohols.

In a further embodiment, b) is selected from the alcohols of the general formulae (4) or (5) of EP 761780 A2, p. 4 described previously, in their ethoxylated form.

c) Hyperbranched polymer HB

The polymers according to the invention comprise, in polymerized-in form, at least one hyperbranched polymer HB with functional groups, where, for the average number f of functional groups per molecule of the hyperbranched polymer, 3<f<100 applies, with the proviso that the hyperbranched polymer is not selected from hyperbranched polyetherpolyols.

Preferred hyperbranched polymers HB are selected from in each case hyperbranched
c1) polyureas
c2) polycarbonates, polyestercarbonates, polyethercarbonates
c3) polyesters, polyetheresters,
c4) polyether ester carbonates,
c5) polyurethanes,
c6) polyisocyanurates,
c7) polyamides, polyester amides
c8) polyamines, polyester amines, polyether amines,
where, for the average number f of the functional groups per molecule of the hyperbranched polymer, 3<f<50 applies, further preferably 3<f<20.

The aforementioned hyperbranched polymers HB are different from hyperbranched polyetherpolyols as described for example in U.S. Pat. No. 3,932,532, DE 10307172, WO 00/56802, WO 2009/101141, Nishikubo et al., Polymer Journal 2004, 36 (5) 413 or Chen et. al, J. Poly. Sci. Part A: Polym. Chem. 2002, 40, 1991, and different from polyglycerol as described for example in WO 2004/074346, DE 19947631, DE 10211664.

The hyperbranched polymers HB can comprise ether groups and hydroxyl groups, but also comprise heteroatoms in groups different from ether and hydroxyl groups, for example in urea, carbonate, ester, urethane, isocyanurate, amide or amino groups.

The hyperbranched polymers HB to be condensed-in preferably comprise end groups selected from hydroxyl, amino, isocyanate, carboxylic acid and carbonyl chloride groups.

The polymers according to the invention can comprise hyperbranched polyetherpolyols and polyglycerol in addition to the aforementioned hyperbranched polymers HB, but not instead of them.

As regards the definition of dendrimeric and hyperbranched polymers, see also P. J. Flory, J. Am. Chem. Soc. 1952, 74, 2718 and H. Frey et al., Chem. Eur. J. 2000, 6, No. 14, 2499.

The hyperbranched polymers c) used according to the invention preferably have a degree of branching (DB) per molecule of from 10 to 100%, preferably 10 to 90% and in particular 20 to 80%. The degree of branching (DB) is the average number of dendritic linkages plus the average number of end groups per molecule, divided by the sum of the average number of dendritic, linear and terminal linkages, multiplied by 100. For the definition of the “degree of branching”, reference is made to H. Frey et al., Acta Polym. 1997, 48.

Within the context of the present invention, the term “hyperbranched polymers” generally comprises polymers which are characterized by a branched structure and a high functionality. Within the context of the invention the “hyperbranched polymers” include dendrimers, hyperbranched polymers and structures derived therefrom.

“Dendrimers” are molecularly uniform macromolecules with a highly symmetrical structure. Dendrimers are derived structurally from star polymers, the individual chains in each case being branched for their part in a star-like manner. They are formed starting from small molecules by means of a continually repeating reaction sequence, during which ever higher branches result, at the ends of which are located in each case functional groups which are in turn the starting point for further branches.

Thus, with each reaction step, the number of monomer end groups increases, ultimately producing a spherical tree structure. A characteristic feature of the dendrimers is the number of reaction steps carried out for their build-up (generations). On account of their uniform build-up, dendrimers usually have a defined molar mass.

Particularly suitable hyperbranched polymers c) are both molecularly and structurally nonuniform hyperbranched polymers which have side chains of differing length and branching, and also a molar mass distribution.

In a preferred embodiment of the invention, the hyperbranched polymers c) are thus not selected from dendrimers.

In particular, so-called ABₓ monomers are suitable for the synthesis of hyperbranched polymers. These have two different functional groups A and B which are able to react with one another to form a linkage. The functional group A is present here only once per monomer and the functional group B is present two or more times. The reaction of said ABₓ monomers with one another essentially produces uncrosslinked polymers with a regular arrangement of branching points. The polymers have virtually exclusively B groups at the chain ends. Details can be found for example in Journal of Molecular Science, Rev. Macromol. Chem. Phys., C37(3), 555-579 (1997).

The term “functional groups” stands for atomic groups in the hyperbranched polymers HB which are able to participate in a chemical reaction, for example in the course of a polymer-analogous functionalization of the hyperbranched polymer HB. Examples of such functional groups are free OH groups, isocyanate groups, carbamoyl groups.

Preferably, as well as the groups resulting during their synthesis (e.g. in the case of hyperbranched polyurethanes, urethane and/or urea groups, and/or further groups arising from the reaction of isocyanate groups; in the case of hyperbranched polyamides, amide groups), the hyperbranched polymers c) have at least four further functional groups. The maximum number of these functional groups is generally not critical. However, it is often not more than 100. Preferably, the fraction of functional groups per molecule is 4 to 100, particularly preferably 5 to 30, and in particular 6 to 20.

According to the invention, the hyperbranched polymer HB preferably has a number-average molecular weight Mₙ of at least 300 g/mol. The number-average molecular weight Mₙ of the hyperbranched polymer is particularly preferably from 500 g/mol to 20 000 g/mol. Weight-average M_{w} molecular weights of the hyperbranched polymer are preferably from 1000 to 100 000 g/mol.

c1) Hyperbranched Polyureas

Hyperbranched polyureas are generally known and their preparation processes are described in detail for example in WO 2003/066702, WO 2005/075541 and WO 2005/044897.

Hyperbranched polyureas suitable according to the invention are also in particular those described in the patent application PCT/EP2010/067978. Reference is hereby made to this disclosure in its entirety.

Within the context of the present invention, the term “polyurea” comprises polymers which, in addition to urea groups, can also have urethane groups, allophanate groups, biuret groups and further groups, such as, for example, amine groups.

The urethane groups are preferably O-alkylurethane groups, where the alkyl radical has 1 to 18 carbon atoms. Preferably, the O-alkylurethane groups are obtainable by reacting an isocyanate group with a monoalcohol which has been used as blocking agent.

Preference is given to hyperbranched polyureas which have a weight-average molecular weight M_{w} in the range from about 500 to 100 000 g/mol, preferably 1000 to 50 000 g/mol. The determination of M_{w} takes place in most cases by gel permeation chromatography. Preferably, the determination is carried out as described in the examples.

Hyperbranched polyureas are accessible in different ways, thus, for example, by directly reacting urea with polyamines and/or by reacting dialkyl carbonates with polyamines. However, preferred hyperbranched polyureas are accessible by reacting a blocked polyisocyanate with polyamines. Further preparation processes are described, e.g. WO 2005/044897 describes the synthesis of hyperbranched polyureas of carbonates (e.g. diethyl carbonate; A₂ monomer) and polyfunctional amines (e.g. triamines; B₃ monomers), or WO05075541 describes the synthesis of hyperbranched polyureas of urea or urea derivatives (A₂ monomers) and polyfunctional amines (e.g. triamines; B₃ monomers).

Hyperbranched polyurea c1) is preferably obtainable by a process comprising the reaction of an at least difunctional blocked di- or polyisocyanate with at least one at least difunctional primary and/or secondary amine with elimination of the blocking agent to give the polyurea.

The at least difunctional blocked di- or polyisocyanates can be prepared, for example, from the reaction of di- or polyisocyanates with aliphatic, araliphatic or aromatic alcohols, preferably monoalcohols. Furthermore, they can be synthesized, for example, by reacting primary amines with alcohol and urea according to EP-A-18586, by reacting primary amines with O-alkyl carbamates according to EP 18588 or EP-A-28338, by reacting primary amines with dimethyl carbonate according to EP-A-570071 or also by reacting formamides with dimethyl carbonate or primary amines with methyl formate according to EP-A-609786. In general, it is also possible to use di- or polyisocyanates which are produced as starting materials or intermediates in the synthesis of phosgene-free prepared di- or polyisocyanates according to the documents EP 355443, EP 566925, EP 568782 or DE 19820114.

In the reaction of the di- or polyisocyanates with the di- or polyamines to give the hyperbranched polyureas, the reversibility of the reaction between isocyanate and alcohol, compared with the irreversibility of the reaction between isocyanate and amine under the given reaction conditions is utilized in order to control a targeted molecule build-up. The alcohol is used here in principle as blocking agent for the isocyanate group, i.e. as moderator for the high reactivity of the isocyanate with the amine. Suitable blocking agents are monoalcohols or blocking reagents, preferably monoalcohols. Suitable monoalcohols are preferably linear or branched aliphatic monoalcohols, such as methanol, ethanol, propanol, butanol, pentanol, hexanol, heptanol, octanol, isopropanol, isobutanol or 2-ethyl-1-hexanol or araliphatic monoalcohols, such as benzyl alcohol or phenylethanol. Particular preference is given to the linear or branched aliphatic monoalcohols and also benzyl alcohol. Linear aliphatic monoalcohols having 1 to 18, preferably 1 to 6, carbon atoms are especially preferred.

In a further embodiment, the starting material is at least difunctional blocked di- or polyisocyanates, the NCO groups of which are blocked with so-called blocking reagents, as are described in the prior art. These blocking reagents are characterized in that they ensure a thermally reversible blocking of the isocyanate groups at temperatures generally below 160° C.

Consequently, blocking agents of this type are used for the modification of isocyanates which are used in thermally curable single-component polyurethane systems. Preferably, the blocking reagents used are phenols, caprolactam, 1H-imidazole, 2-methylimidazole, 1,2,4-triazole, 3,5-dimethylpyrazole, malonic acid dialkyl ester, acetanilide, acetone oxime or butanone oxime. The reaction with the di- or polyamine to give the hyperbranched polyurea also takes place here with the elimination of the blocking agent. Consequently, the NCO groups blocked with monoalcohols or with blocking reagents are referred to hereinbelow as “capped NCO groups”.

The hyperbranched polyurea is terminated after the reaction, i.e. without modification, either with amino groups or with capped NCO groups.

The hyperbranched polyureas dissolve well in polar solvents, for example in alcohols, such as methanol, ethanol, butanol, alcohol/water mixtures, esters such as ethyl acetate and butyl acetate, furthermore in dimethylformamide, dimethylacetamide, N-methylpyrrolidone, ethylene carbonate or propylene carbonate.

Besides urea groups, a hyperbranched polyurea c1) also has at least three, preferably at least six, more preferably at least eight, functional groups.

The number of functional groups is in principle not limited upwardly, although products with a very large number of functional groups can have undesired properties, such as, for example, high intrinsic viscosity or poor solubility.

The hyperbranched highly functional polyureas c1) of the present invention preferably have, per molecule, on average not more than 100, further preferably not more than 50, functional groups different from urea groups. The at least difunctional primary and/or secondary amines used in the preparation of the hyperbranched polyureas c1) are selected from compounds which carry at least two reactive amine groups.

Compounds with at least two reactive amine groups are, for example, ethylenediamine, N-alkylethylenediamine, propylenediamine, 2,2-dimethyl-1,3-propanediamine, N-alkylpropylenediamine, butylenediamine, N-alkylbutylenediamine, hexamethylenediamine, N-alkylhexamethylenediamine, tolylenediamine, diaminodiphenylmethane, diaminodicyclohexylmethane, phenylenediamine, cyclohexyldiamine, diaminodiphenylsulfone, isophoronediamine, 2-butyl-2-ethyl-1,5-pentamethylenediamine, 2,2,4- or 2,4,4-trimethyl-1,6-hexamethylenediamine, 2-aminopropylcyclohexylamine, 3(4)-aminomethyl-1-methylcyclohexylamine, 1,4-diamino-4-methylpentane, amine-terminated polyoxyalkylenepolyols (so-called Jeffamines), aminated polytetramethylene glycols, N-aminoalkylpiperidines, ammonia, bis(aminoethyl)amine, bis(aminopropyl)amine, bis(aminobutyl)amine, bis(aminopentyl)amine, bis(aminohexyl)amine, tris(aminoethyl)amine, tris(aminopropyl)amine, tris(aminohexyl)amine, trisaminohexane, 4-aminomethyl-1,8-octamethylenediamine, N′-(3-aminopropyl)-N,N-dimethyl-1,3-propanediamine, trisaminononane or melamine. Furthermore, it is also possible to use any desired mixtures of at least two of the stated compounds.

Preferred at least difunctional primary and/or secondary amines are at least difunctional primary amines, particularly preferably difunctional aliphatic primary amines, in particular isophoronediamine.

Suitable di- or polyisocyanates are the aliphatic, cycloaliphatic, araliphatic and aromatic di- or polyisocyanates known according to the prior art and specified below by way of example. To be mentioned here are, preferably, 4,4′-diphenylmethane diisocyanate, the mixtures of monomeric diphenylmethane diisocyanates and oligomeric diphenylmethane diisocyanates (polymer-MDI), tetramethylene diisocyanate, tetramethylene diisocyanate trimers, hexamethylene diisocyanate, hexamethylene diisocyanate trimers, isophorone diisocyanate trimer, 4,4′-methylenebis(cyclohexyl) diisocyanate, xylylene diisocyanate, tetramethylxylylene diisocyanate, dodecyl diisocyanate, lysine alkyl ester diisocyanate, where alkyl is C1 to C10, 1,4-diisocyanatocyclohexane or 4-isocyanatomethyl-1,8-octamethylene diisocyanate. Of particularly preferred suitability for building up the polyureas c1) are di- or polyisocyanates which have NCO groups of varying reactivity. Mention may be made here of 2,4-tolylene diisocyanate (2,4-TDI), 2,4′-diphenylmethane diisocyanate (2,4′-MDI), triisocyanatotoluene, isophorone diisocyanate (IPDI), 2-butyl-2-ethylpentamethylene diisocyanate, 2,2,4- or 2,4,4-trimethyl-1,6-hexamethylene diisocyanate, 2-isocyanatopropylcyclohexyl isocyanate, 3(4)-isocyanatomethyl-1-methylcyclohexyl isocyanate, 1,4-diisocyanato-4-methylpentane, 2,4′-methylenebis(cyclohexyl) diisocyanate and 4-methylcyclohexane 1,3-diisocyanate (HTDI). Also of suitability for building up the polyureas are isocyanates, the NCO groups of which are initially equally reactive, but in which, as a result of the first addition of a reactant to one NCO group, a drop in reactivity in the case of the second NCO group can be induced. Examples thereof are isocyanates, the NCO groups of which are coupled via a delocalized p-electron system, e.g. 1,3- and 1,4-phenylene diisocyanate, 1,5-naphthylene diisocyanate, diphenyl diisocyanate, tolidine diisocyanate or 2,6-tolylene diisocyanate. It is also possible to use, for example, oligo- or polyisocyanates which can be prepared from the aforementioned di- or polyisocyanates or mixtures thereof by linkage by means of urethane, allophanate, urea, biuret, uretdione, amide, isocyanurate, carbodiimide, uretonimine, oxadiazinetrione or iminooxadiazinedione structures.

Di- or polyisocyanates that are specifically preferably suitable for the build-up of the polyureas are oligo- or polyisocyanates which can be prepared from aliphatic, cycloaliphatic, araliphatic and aromatic, preferably aliphatic, di- or polyisocyanates through linkage by means of urethane, allophanate, urea, biuret, uretdione, amide, isocyanurate, carbodiimide, uretonimine, oxadiazinetrione or iminooxadiazinedione structures, preferably by means of isocyanurate structures. Usually, these oligo- or polyisocyanates have an average NCO functionality of from 2.1 to 4.9, preferably 2.9 to 4.4, in particular from 3.4 to 3.9. The average molar mass is in most cases 300 to 3000 g/mol, preferably 400 to 1500 g/mol, in particular 500 to 800 g/mol.

During the preparation of the hyperbranched highly functional polyureas c1), it is necessary to adjust the ratio of compounds having at least two amine groups that are reactive with capped NCO groups to the capped isocyanate in molar terms such that the resulting simplest conceivable condensation product (termed hereinbelow condensation product (A)) comprises on average either one capped NCO group and more than one group that is reactive with the capped NCO group, or one group that is reactive with capped NCO groups and more than one capped NCO group. The simplest structure of the condensation product (A) of one capped di- or polyisocyanate (X) and a di- or polyamine (Y) produces here the arrangement XYₙ or XₙY, where n is generally a number from 1 to 6, preferably from 1 to 4, particularly preferably from 1 to 3. The reactive group, which results in the process as the only group, is generally termed hereinbelow “focal group”.

If, for example, in the case of the preparation of the simplest condensation product (A) from a capped diisocyanate and a divalent amine, the conversion ratio is 1:1, then a molecule of the type XY results. In the case of the preparation of the condensation product (A) from a capped diisocyanate and a trivalent amine with a conversion ratio of 1:1, a molecule of the type XY₂ results. The focal group here is a capped isocyanate group. In the case of the preparation of the condensation product (A) from a capped diisocyanate and a tetravalent amine likewise with the conversion ratio 1:1, a molecule of the type XY₃ results. The focal group here is a capped isocyanate. Furthermore, the preparation of the condensation product (A) can take place for example also from a capped diisocyanate and a trivalent component that is reactive with the capped diisocyanate, the conversion ratio being, in molar terms, 2:1. Here, a molecule of the type X₂Y results, and the focal group here is an amine. If difunctional compounds, e.g. having two capped isocyanate groups or having two amine groups, are additionally added to the components, then this brings about a lengthening of the chains. Again, a molecule of the type X₂Y results, and the focal group is a capped isocyanate. The reaction product (A) is preferably not isolated. Preferably, in the further course of the process, the conversion of the reaction products (A) to the hyperbranched polyurea (P) takes place directly.

The conversion to the condensation product (A) and to the polycondensation product (P) usually takes place at a temperature from 0 to 250° C., preferably at 60 to 160° C., without dilution or in solution. In this connection, in general, it is possible to use all solvents which are inert toward the particular starting materials. Preference is given to using organic solvents, such as, for example, decane, dodecane, benzene, toluene, chlorobenzene, xylene, dimethylformamide, dimethylacetamide or solvent naphtha. In a preferred embodiment, the condensation reaction is carried out without dilution. The capping agent which is released during the reaction with the amine, for example the alcohol used for the urethanization, can be removed from the reaction equilibrium by distillation, optionally under reduced pressure, in order to increase the rate of the reaction.

In a further preferred embodiment, the alcohol used for the blocking is used as solvent for the reaction. In this case, the urethane component is introduced as initial charge as a solution in the alcohol, and the amine component is added in the corresponding ratio. Upon increasing the temperature, the alcohol bonded as urethane is displaced by the amine component, and the urea according to the invention is formed. The alcohol component present in excess also functions as solvent for the ureas formed.

To increase the rate of the reaction, it is also possible to add catalysts or catalyst mixtures. Suitable catalysts are generally compounds which catalyze urethane reactions, for example amines, ammonium compounds, organoaluminum, organotin, organozinc, organotitanium, organozirconium or organobismuth compounds. For example, diazabicyclooctane (DABCO), diazabicyclononene (DBN), diazabicycloundecene (DBU), imidazoles, such as imidazole, 1-methylimidazole, 2-methylimidazole, 1,2-dimethylimidazole, titanium tetrabutylate, dibutyltin oxide, dibutyltin dilaurate, tin dioctoate, zirconium acetylacetonate or mixtures thereof can be used. The addition of the catalyst takes place generally in an amount from 50 to 10 000, preferably from 100 to 5000 ppm by weight, based on the amount of isocyanate used. In addition, it is also possible to control the intermolecular polycondensation reaction either by adding the suitable catalyst, and also through selection of a suitable temperature.

Furthermore, the average molecular weight of the polymer can be adjusted via the composition of the starting components and via the residence time. The condensation products (A) and/or the polycondensation products which have been prepared at elevated temperature are usually stable over a prolonged period at room temperature. On account of the nature of the condensation products (A) it is possible for polycondensation products to result from the condensation reaction that have different structures which have branches but no crosslinkages. Furthermore, the polycondensation products have either a capped isocyanate group as focal group and more than two groups that are reactive with capped isocyanate groups, or else one group that is reactive with capped isocyanate as focal group and more than two capped isocyanate groups. The number of reactive groups arises here from the nature of the condensation products (A) used and the degree of polycondensation.

There are various options for terminating the intermolecular polycondensation reaction. For example, the temperature can be reduced to a range in which the reaction comes to a standstill and the product (A) or the polycondensation product is storage-stable. In a preferred embodiment, as soon as, on account of the intermolecular reaction of the condensation product (A), a polycondensation product with the desired degree of polycondensation is present, a product with groups that are reactive toward the focal group of (P) is added to the product in order to terminate the reaction. Thus, in the case of a capped NCO group as focal group, for example a mono-, di- or polyamine can be added. In the case of an amine as focal group, a mono-, di- or polyurethane, a mono-, di- or polyisocyanate, an aldehyde, ketone or an acid derivative that is reactive with amine, for example, can be added to the product (P).

The preparation of the hyperbranched polyureas takes place in most cases in a pressure range from 2 mbar to 20 bar, preferably at atmospheric pressure, in reactors or reactor cascades which are operated batchwise, semicontinuously or continuously. By means of the aforementioned adjustment of the reaction conditions and optionally through the selection of the suitable solvent, the products according to the invention can be further processed after the preparation without further purification.

Hyperbranched polyureas suitable according to the invention are also the hyperbranched polyureas described in WO 2006/087227 on page 9, line 5 to page 14, line 3.

A particular embodiment of the present invention comprises polymers P comprising, in polymerized-in form,
a) at least one polyisocyanate
b) at least one alcohol of the general formula I

R¹O—R²ₙOH (I)
where
R¹ is selected from C₆-C₄₀-alkyl, C₆-C₄₀-alkenyl, C₃-C₁₀-cycloalkyl, C₆-C₃₀-aryl, C₇-C₄₀-arylalkyl,
R² is selected from C₂-C₁₀-alkylene, C₆-C₁₀-arylene, C₇-C₁₀-arylalkylene,
n is selected from 0 to 200
c) at least one hyperbranched polymer HB with functional groups, where, for the average number f of functional groups per molecule of the hyperbranched polymer, 3<f<100 applies and where HB is a hyperbranched polyurea,
d) optionally at least one compound different from b) and c) and having a molecular weight of at least 300 g/mol comprising
   i. at least two OH groups and
   ii. at least two groups selected from ether groups and ester groups,
e) optionally further compounds different from b) to d) and having 1 to 10 groups that are reactive toward isocyanate groups per molecule.

The polymers according to the invention which comprise as c) a hyperbranched polyurea in polymerized-in form, may be used for increasing the water binding capacity in an aqueous, in particular cosmetic, preparation. They can also be used for increasing the water binding capacity of the skin (i.e. as so-called moisturizer).

c2) Hyperbranched Polycarbonates

Hyperbranched polycarbonates are generally known.

WO 2006/089940 discloses water-emulsifiable hyperbranched polycarbonates which are reacted at least partially directly with a monofunctional polyalkylene oxide polyether alcohol.

WO 2005/075565 discloses the reaction of a hyperbranched polycarbonate with a functionalization reagent which is able to react with the OH and/or carbonate groups or carbamoyl groups of the polycarbonate.

WO 2007/134736 and WO 2008/009516 disclose the reaction of a hyperbranched polycarbonate with a functionalization reagent which is able to react with the OH and/or carbonate groups or carbamoyl groups of the polycarbonate. By way of example, the reaction with compounds comprising anhydride groups is specified, such that polycarbonates comprising acid groups can be obtained.

The hyperbranched polycarbonates described in the aforementioned disclosures are suitable according to the invention as hyperbranched polycarbonates c2).

WO 2010/130599 describes amphiphiles which comprise hyperbranched polycarbonates in incorporated form.

In particular, the hyperbranched polycarbonates described in WO 2010/130599, page 5, line 29 to page 16, line 36 and also described by way of example in Synthesis Examples A.1 to A.4 are suitable according to the invention as hyperbranched polycarbonates c2).

A particular embodiment of the present invention comprises polymers P comprising, in polymerized-in form,
a) at least one polyisocyanate
b) at least one alcohol of the general formula I

R¹O—R²ₙOH (I)
where
R¹ is selected from C₆-C₄₀-alkyl, C₆-C₄₀-alkenyl, C₃-C₁₀-cycloalkyl, C₆-C₃₀-aryl, C₇-C₄₀-arylalkyl,
R² is selected from C₂-C₁₀-alkylene, C₆-C₁₀-arylene, C₇-C₁₀-arylalkylene,
n is selected from 0 to 200
c) at least one hyperbranched polymer HB with functional groups, where, for the average number f of functional groups per molecule of the hyperbranched polymer, 3<f<100 applies and where HB is a hyperbranched polycarbonate obtainable by
   A) preparation of a condensation product (K) by reacting an organic carbonate (A) or a phosgene derivative with an alcohol (B1) which has at least three hydroxy groups, and
   B) intermolecular reaction of K to give the hyperbranched polycarbonate, with the proviso that the hyperbranched polymer is not selected from hyperbranched polyetherpolyols,
d) optionally at least one compound different from b) and c) and having a molecular weight of at least 300 g/mol comprising
   i. at least two OH groups and
   ii. at least two groups selected from ether groups and ester groups,
e) optionally further compounds different from b) to d) and having in the range from 1 to 10 groups that are reactive toward isocyanate groups per molecule.

A particular embodiment of the present invention comprises polymers P, where the hyperbranched polycarbonate is obtainable by
A) preparation of a condensation product (K) by reacting an organic carbonate or a phosgene derivative with an alcohol (B1) comprising at least three OH groups, and
B) subsequent reaction of the condensation product (K) to give the hyperbranched polycarbonate,
   where the quantitative ratio of the OH groups to the carbonate or phosgene groups is selected such that the condensation product (K) has on average either one carbonate or carbamoyl chloride group and more than one OH group, or one OH group and more than one carbonate or carbamoyl group.

In one embodiment of the invention, the alcohol (B1) comprising at least 3 OH groups is or comprises a polyetherpolyol.

Also in accordance with the invention are polymers where the condensation product K underlying the hyperbranched polymer HB c) comprises at least one polyetherol in condensed-in form which is obtainable by the alkoxylation of at least trifunctional alcohols with C₂-C₄ alkylene oxide.

The present invention further provides the use of the polymers according to the invention which comprise, as c), a hyperbranched polycarbonate in polymerized-in form, for improving the skin feel.

The present invention further provides the use of the polymers according to the invention which comprise, as c), a hyperbranched polycarbonate in polymerized-in form, for solubilizing active ingredients.

c3) Hyperbranched Polyesters

Hyperbranched polyesters are generally known.

Of suitability as c3) according to the invention are, for example, the hyperbranched polyesters comprising dicarboxylic acid units and trifunctional alcohols disclosed in WO 2009/047210. The dicarboxylic acid units with C₃-C₄₀ alkyl radicals or alkenyl radicals used are substituted succinic acid units, and the trifunctional alcohols used are, for example, glycerol, trimethylolpropane, pentaerythritol and alkoxylated derivatives thereof.

Of suitability as c3) according to the invention are also the hyperbranched polyesters disclosed in WO 2007/068632 which are obtainable by reacting dicarboxylic acids having polyisobutene groups with trifunctional alcohols such as glycerol, trimethylolpropane, pentaerythritol and alkoxylated derivatives thereof.

Hyperbranched polyesters c3) that are particularly suitable according to the invention comprise, in condensed-in form, at least one hydrophobic dicarboxylic acid selected from aliphatic C₁₀-C₃₂ dicarboxylic acids, dicarboxylic acids having at least one polyisobutylene group and succinic acid units having at least one C₃-C₄₀ group, and at least one trifunctional alcohol selected from glycerol, trimethylolethane, trimethylolpropane, bis(trimethylolpropane), pentaerythritol and alkoxylated derivatives thereof.

The hyperbranched polyesters defined in claims 1 to 6 and also on page 7, line 17 to page 17, line 36 of the patent application PCT/EP2010/069680 are particularly suitable according to the invention.

Also of suitability as c3) according to the invention are the hyperbranched polyesters described in WO 2007/125028, page 1, line 7 to page 2, line 8, on page 12, line 20 to page 18, line 23 and also in Examples (a.1) to (a.6).

A particular embodiment of the present invention comprises polymers P comprising, in polymerized-in form,
a) at least one polyisocyanate
b) at least one alcohol of the general formula I

R¹O—R²ₙOH (I)
where
R¹ is selected from C₆-C₄₀-alkyl, C₆-C₄₀-alkenyl, C₃-C₁₀-cycloalkyl, C₆-C₃₀-aryl, C₇-C₄₀-arylalkyl,
R² is selected from C₂-C₁₀-alkylene, C₆-C₁₀-arylene, C₇-C₁₀-arylalkylene,
n is selected from 0 to 200
c) at least one hyperbranched polymer HB with functional groups, where, for the average number f of functional groups per molecule of the hyperbranched polymer, 3<f<100 applies, and where HB is a hyperbranched polyester which comprises, in condensed-in form, at least one hydrophobic dicarboxylic acid selected from aliphatic C₁₀-C₃₂ dicarboxylic acids, dicarboxylic acids having at least one polyisobutylene group and succinic acid units having at least one C₃-C₄₀ group, and at least one trifunctional alcohol selected from glycerol, trimethylolethane, trimethylolpropane, bis(trimethylolpropane), pentaerythritol and alkoxylated derivatives thereof,
d) optionally at least one compound different from b) and c) and having a molecular weight of at least 300 g/mol comprising
   i. at least two OH groups and
   ii. at least two groups selected from ether groups and ester groups,
e) optionally further compounds different from b) to d) and having in the range from 1 to 10 groups that are reactive toward isocyanate groups per molecule.
   c5) Hyperbranched Polyurethanes

Within the context of this invention, the term “polyurethanes” comprises not only those polymers whose repeat units are joined together by urethane groups, but quite generally polymers which, in addition to urethane groups, comprise further groups such as urea, allophanate, biuret, carbodiimide, amide, uretonimine, uretdione, isocyanurate or oxazolidone (oxazolidinone) groups (see for example Kunststofftaschenbuch [Plastics handbook], Saechtling, 26^{th} edition, p. 491ff, Carl-Hanser-Verlag, Munich 1995). According to the invention, the term “polyurethanes” comprises in particular polymers which also have urea groups as well as urethane groups.

Hyperbranched polyurethanes c5) suitable according to the invention are, for example, those described in DE 10322401 A1. Of suitability in particular are those hyperbranched polyurethanes which are obtainable by a process according to any one of claims 1 to 7 of DE 10322401 A1.

Hyperbranched polyurethanes c5) suitable according to the invention are, for example, also those described in EP 1026185 A1. Of suitability in particular are those hyperbranched polyurethanes which are obtainable by a process according to any one of claims 1 to 7 of EP 1026185 A1.

Hyperbranched polyurethanes c5) suitable according to the invention are also the hyperbranched polyurethanes described in WO 2006/087227 on page 9, line 5 to page 14, line 3.

c6) Hyperbranched Polyisocyanurates

A preferred hyperbranched polyisocyanurate c6) is obtainable by the, preferably acid-catalyzed, condensation of tris(hydroxyalkyl) isocyanurate, preferably tris(hydroxyethyl) isocyanurate, polyhydric alcohol, preferably diethylene glycol and water. Preference is given, for example, to polyisocyanurates as described in the European patent application No. 10187941.9, to which reference is hereby made.

c7) Hyperbranched Polyamides

Hyperbranched polyamides are described, for example, in U.S. Pat. No. 4,507,466, U.S. Pat. No. 6,541,600, US 2003055209, U.S. Pat. No. 6,300,424, U.S. Pat. No. 5,514,764 and WO 92/08749, to which reference is hereby made in their entirety.

Polyamides preferred according to the invention are obtainable by procedures as described in WO 2006/087227 on page 14, line 11 to page 17, line 9.

Hyperbranched polyester amides suitable according to the invention are described, for example in WO 99/16810 and WO 00/56804, to which reference is made here in their entirety.

Polyester amides preferred according to the invention and processes for their preparation are described in WO 2006/087227 on page 17, line 13 to page 21, line 29.

c8) Hyperbranched Polyamines

Suitable hyperbranched polymers HB according to the invention are also hyperbranched polyether amines. As is known, polyether amine polyols are obtained from trialkanolamines, such as, for example, triethanolamine, tripropanolamine, triisopropanolamine, optionally also in a mixture with mono- or dialkanolamines, by etherifying these monomers with catalysis, e.g. acidic or basic catalysis, with the elimination of water. The preparation of hyperbranched polyether amines suitable according to the invention is described, for example, in U.S. Pat. No. 2,178,173, U.S. Pat. No. 2,290,415, U.S. Pat. No. 2,407,895 and DE 4003243.

Hyperbranched polyether amines suitable according to the invention are, for example, the trialkanolamine polyethers described in DE 4003243 A1, page 2, lines 40-51 and patent claims 1 and 2.

Hyperbranched polyether amines suitable according to the invention are for example the polyether amine polyols based on trialkanol monomers and optionally further monomer types described in WO 2009/047269. Preferred hyperbranched polyether amines of WO 2009/047269 are composed of triethanolamine monomers, triisopropanolamine monomers and/or tripropanolamine monomers and are obtainable by acid- or base-catalyzed condensation of the aforementioned monomers, in particular of triethanolamine. Reference is made to the disclosure of WO 2009/047269 in its entirety.

A particular embodiment of the present invention comprises polymers P comprising, in polymerized-in form,
a) at least one polyisocyanate
b) at least one alcohol of the general formula I

R¹O—R²ₙOH (I)
where
R¹ is selected from C₆-C₄₀-alkyl, C₆-C₄₀-alkenyl, C₃-C₁₀-cycloalkyl, C₆-C₃₀-aryl, C₇-C₄₀-arylalkyl,
R² is selected from C₂-C₁₀-alkylene, C₆-C₁₀-arylene, C₇-C₁₀-arylalkylene,
n is selected from 0 to 200
c) at least one hyperbranched polymer HB with functional groups, where, for the average number f of functional groups per molecule of the hyperbranched polymer, 3<f<100 applies, and where HB is a hyperbranched polyamine obtainable by condensation of trialkanolamine,
d) optionally at least one compound different from b) and c) and having a molecular weight of at least 300 g/mol comprising
   i. at least two OH groups and
   ii. at least two groups selected from ether groups and ester groups,
e) optionally further compounds different from b) to d) and having in the range from 1 to 10 groups that are reactive toward isocyanate groups per molecule.

The polymers according to the invention which comprise, as c), a hyperbranched polyether amine in polymerized-in form, may be used as auxiliary for silicone depositioning.

The present invention further provides the use of the polymers according to the invention which comprise, as c), a hyperbranched polyether amine in polymerized-in form, for increasing the salt stability of aqueous preparations.

The invention further provides the use of the polymers according to the invention which comprise, as c), a hyperbranched polyether amine in polymerized-in form, for improving the skin feel.

Further suitable hyperbranched polyamines are hyperbranched polyester amines described in WO 2006/087227 on page 21, line 31 to page 25, line 2.

d) Polyols Different from b) and c)

Optionally, the polymers according to the invention comprise, in polymerized-in form, at least one compound d) different from b) and c) and having a molecular weight of at least 300 g/mol, preferably at least 1200 g/mol.

Compound d) comprises, per molecule, at least two OH groups and at least two groups selected from ether groups and ester groups. Polyol d) is thus selected from polyetherols, polyesterols and polyetheresterols.

In one embodiment of the invention, polyol d) has a number-average molecular weight Mₙ of from 1500 to 20 000 g/mol, preferably from 4000 to 12 000 g/mol.

Suitable polyols d) are, for example, the polymerization products of ethylene oxide, their mixed- or graft-polymerization products, and also the polyethers obtained by condensation of polyhydric alcohols or mixture thereof and the polyethers obtained by ethoxylation of polyhydric alcohols, amides, polyamides and amino alcohols. Examples thereof are, for example, polyethylene glycols, addition products of ethylene oxide onto trimethylolpropane, EO-PO block copolymers, OH-terminated polyesters such as, for example, those of the polyfunctional polycaprolactone type.

Preferred polyols d) are polyetherpolyols. These are polyols which comprise, per molecule, at least two OH groups and at least two functions —O— (ether groups). These polyetherpolyols are generally so strongly hydrophilic that they are water-soluble at room temperature (20° C.).

Particularly preferred polyols d) comprise, per molecule, on average from 30 to 450 CH₂CH₂—O— units (EO units). Preferred compounds d) are thus polyols of the general formula HO—(CH₂—CH₂—O)ₙ—H, where n can assume the values 30 to 450. These are usually condensation products of ethylene oxide with ethylene glycol or water. Preferred polyethylene glycols d) have a molecular weight Mₙ in the range from 1500 to 20 000 g/mol, preferably from 4000 to 12 000 g/mol.

Suitable compounds d) are also ethylene oxide-propylene oxide block copolymers, such as, for example, EO-PO block copolymers of the general formula HO-(EO)ₘ—(PO)ₙ-(EO)_{O}—H, where m and o independently of one another, are integers in the range from 10 to 100, preferably from 20 to 80, n is an integer in the range from 5 to 50, preferably from 20 to 40, and where m, n and o are selected such that HO-(EO)ₘ—(PO)ₙ-(E_{O})_{O}—H is water-soluble.

In one embodiment, the polyetherols d) have a molecular weight Mₙ in the range from 1500 g/mol to 15 000 g/mol.

In a further embodiment, the polyetherols d) have a molecular weight Mₙ in the range from 4000 g/mol to 12 000 g/mol.

In a preferred embodiment, the polyetherols d) have a molecular weight Mₙ in the range from 6000 g/mol to 12 000 g/mol.

In a further preferred embodiment, the polyetherols d) have a molecular weight Mₙ in the range from 6000 g/mol to 10 000 g/mol.

In one embodiment, the polyetherols d) have a molecular weight Mₙ of about 10 000 g/mol.

In a further particularly preferred embodiment, the polyetherols d) have a molecular weight Mₙ of about 6000 g/mol. A suitable polyetherol is, for example, the product available under the trade name Pluriol® E 6000.

In a further particularly preferred embodiment, the polyetherols d) have a molecular weight Mₙ of about 9000 g/mol.

In one embodiment of the invention, for the preparation of the polymers according to the invention, based on the total amount of all polymerized compounds, at most 5% by weight, preferably less than 1% by weight, further preferably no compounds d) are used.

In this way, polymers with a particularly low melt viscosity are obtained which can be handled easily in pure form. The viscosity increase arises only after the addition of water. Thus, firstly, an easy-to-handle thickener preproduct is obtained which, only upon the addition of water, i.e. for example upon use in a cosmetic preparation, has a thickening effect.

e) Further Compounds with Groups that are Reactive Toward NCO Per Molecule

The polymers according to the invention optionally comprise, in polymerized-in form, further compounds e) different from a) to d) and having, per molecule, in the range from 1 to 10, preferably from 1 to 9, groups that are reactive toward isocyanate groups. Compounds with groups that are reactive toward isocyanate groups are preferably selected from compounds with hydroxyl groups, such as, for example, alcohols, compounds with amino groups, such as, for example, amines and compounds with hydroxyl groups and amino groups, such as, for example, amino alcohols.

Examples of compounds e) having up to 8 hydroxyl groups per molecule are disclosed, for example, in EP 1584331A1, paragraph [0039], to which reference is hereby made. Suitable compounds with amino groups are, for example, ethylenediamine, diethylenetriamine and propylenediamine.

Suitable compounds with hydroxyl groups and amino groups are, for example, ethanolamine and diethanolamine.

### Preparation Processes

The polymers according to the invention comprise the components a), b) and c) preferably in the following ratios (mol to mol):

If the polymers according to the invention comprise compound d) in polymerized-in form:

a:b from 10:1 to 1:1.9; preferably 5:1 to 1:1
b:c from 25:1 to 1:1; preferably 10:1 to 1.5:1
a:d from 10:1 to 1:1.9; preferably 5:1 to 1:1

If the polymers according to the invention comprise no d) in polymerized-in form:

a:b from 1.5:1 to 1:1.9; preferably 1.2:1 to 1:1.5
b:c from 25:1 to 1:1; preferably 10:1 to 1.5:1

Compound e) is preferably polymerized-in in an amount such that from 0 to 50 mol %, particularly preferably from 0 to 25 mol %, very particularly preferably from 0 to 10 mol %, of all groups of components b) to e) that are reactive toward isocyanate groups originate from e).

In one embodiment, e) is polymerized-in in an amount such that from 0 to 1 mol % of all groups of components b) to e) that are reactive toward isocyanate groups originate from e).

In a further embodiment, no compound e) is polymerized-in.

The present invention further provides also processes for the preparation of the polymers according to the invention. These processes according to the invention are described below. The individual reaction steps are assigned Roman numerals. Steps with higher numerals are carried out after steps with lower numerals.

To prepare the polymers according to the invention, the components a) to e) can be polymerized in the presence of a solvent different from a) to e). Solvent here is understood as meaning a compound inert toward a) to e) but in which the starting compounds a) to e), the intermediates and the polymers are soluble. In the present case, soluble means that at least 1 g of the substance in question is dissolved to give a solution that is clear to the human eye in 1 liter of solvent under standard conditions.

In one embodiment of the invention, the polymers according to the invention are prepared from the compounds a) to e) in solvents selected from xylene, toluene, acetone, tetrahydrofuran (THF), butyl acetate, N-methylpyrrolidone, N-ethylpyrrolidone and mixtures thereof.

In another embodiment of the invention, the polymers according to the invention are prepared from the compounds a) to e) essentially in the absence of solvents. Essentially in the absence of solvents means that, with regard to the total amount of the compounds a) to e), the polymerization is carried out in the presence of less 10% by weight, preferably less than 5% by weight, of a solvent different from a) to e).

To prepare the polymers according to the invention, in principle all catalysts customarily used in polyurethane chemistry are suitable.

Such suitable catalysts and their amount, solvent and type of addition are described, for example, in WO 2009/135856, p. 11, I. 35 to p. 12, I. 42, to which reference is hereby made.

Preferred catalysts are zinc carboxylates, in particular selected from zinc 2-ethylhexanoate, zinc n-octanoate, zinc n-decanoate, zinc neodecanoate, zinc ricinoleate and zinc stearate. Particular preference is given to using zinc neodecanoate.

Suitable catalysts are also alkali(ne earth) metal salts of inorganic acid or of carboxylic acids such as, for example, potassium salts of acetic acid, citric acid, lactic acid, oxalic acid.

According to the invention, it is preferred if all of the substances used in the process are essentially anhydrous. “Essentially anhydrous” means that the water content of all substances used in the process is less than 5% by weight, preferably less than 1% by weight, particularly preferably less than 0.1% by weight, based on the total amount of the respective substance.

Methods of removing water from the substances before they are brought into contact with the NCO-group-comprising substances are customary and known to the person skilled in the art.

In one embodiment of the invention, to prepare the polymers according to the invention,

I) the component d) is introduced as initial charge,
II) the addition of component a) is started,
III) upon reaching an NCO value of preferably at most 50% of the starting value, the addition of component b) is started,
IV) after at least 50, preferably at least 80, particularly preferably at least 90% by weight of b) have been polymerized-in, the addition of component c) is started.

In one embodiment of the invention, to prepare the polymers according to the invention,

I) d) is introduced as initial charge,
II) the addition of a) is started,
II) upon reaching an NCO value in the range from 99.9 to 0.1% of the starting value, preferably from 80 to 5% of the starting value, the addition of b) and c) is started at about the same time.

In a preferred embodiment of the invention, to prepare the polymers according to the invention,

I) d) is introduced as initial charge,
II) the addition of a) is started,
III) upon reaching an NCO value in the range from 99.9 to 0.1% of the starting value, preferably from 80 to 5% of the starting value, the addition of b) is started,
IV) upon reaching an NCO value in the range from 95 to 5% of the starting value, preferably 50 to 5% of the starting value, the addition of c) is started.

Step IV) takes place after step III).

In a further embodiment of the invention, to prepare the polymers according to the invention

I) the component b) is introduced as initial charge,
II) the addition of component a) is started,
III) upon reaching an NCO value in the range from 99.9 to 0.1% of the starting value, preferably from 80 to 5% of the starting value, very particularly preferably from 50 to 5% of the starting value, the addition of component c) is started.

A possible embodiment of the present invention is a process for the preparation of the polymers according to the invention, comprising the steps

I) introduction of b) as initial charge,
II) addition of a),
III) start of the addition of c) when the NCO value is in the range from 99.9 to 0.1%, preferably from 80 to 5%, further preferably from 50 to 5%, of the starting value.

Preferably, the polymer obtainable by this specific embodiment has, based on its total weight, less than 5% by weight, further preferably less than 1% by weight and in particular 0% by weight, of compound d) in polymerized-in form.

The NCO value (isocyanate content) was determined titrimetrically in accordance with DIN 53185.

In a further embodiment of the invention, to prepare the polymers according to the invention,

I) the component d) is introduced as initial charge,
II) the addition of component a) is started,
III) upon reaching an NCO value in the range of preferably at most 50% of the starting value, the components b) and c) are added simultaneously and preferably mixed.

In a further embodiment of the invention, to prepare the polymers according to the invention,

I) the component b) is introduced as initial charge,
II) the addition of component a) is started,
III) upon reaching an NCO value in the range of preferably at most 50% of the starting value, the addition of component c) is started.

The NCO value (isocyanate content) was determined in accordance with DIN 53185.

### Modification of Compound c)

In a preferred embodiment, the hyperbranched polymer HB c) still comprises free functional groups even after the polymerization. Compared with conventional associative thickeners, these bring about an increased solubility of the polymers according to the invention in polar solvents, in particular in alcohols and water. The free OH groups of the polymerized-in compound c) also have a positive influence on the structure and the visual appearance of the preparations comprising the polymers according to the invention.

The present invention provides polymers P according to the invention, where, as a result of the polymerization, in the range from 5 to 95 mol % of the functional groups of the hyperbranched polymer HB present before the polymerization are consumed.

The present invention preferably provides polymers P according to the invention in which 80 mol %, preferably up to 60 mol %, of the functional reactive groups present in the hyperbranched polymers HB before the polymerization are present in unchanged form after the polymerization.

The hyperbranched polymer HB can be modified before the polymerization by reacting at least some of its functional groups. This is possible either by preparing HB in the presence of modifying reagents or by modifying HB after its preparation.

The present invention further provides modified polymers MP1 obtainable by reacting at least some of the functional groups of a polymer P according to the invention with compounds that are reactive toward these functional groups.

The present invention also provides modified polymers MP1 obtainable by the reaction of at least some of the functional groups of the polymerized-in hyperbranched polymer HB of the polymer P according to the invention that are still present after the polymerization with compounds that are reactive toward these functional groups.

Modified polymers MP1 are preferably obtained by reacting the polymer P according to the invention in an additional process step with suitable modifying reagents which are able to react with the functional groups of HP that remain after the polymerization.

The remaining functional groups of the polymerized-in HB can be modified, for example, by adding modifying reagents comprising acid, acid halide or isocyanate groups. A functionalization of the polymerized-in compound c) with acid groups can take place for example by reacting OH groups with compounds comprising anhydride groups. Ester groups can be introduced subsequently, for example by reaction with caprolactone. Here, the length of the ester chains can be controlled via the amount of caprolactone used.

Furthermore, the polymerized-in HB can also be functionalized by reaction with alkylene oxides, for example ethylene oxide, propylene oxide, butylene oxide or mixtures thereof.

The present invention also provides polymers obtainable by functionalization of the polymerized-in compound c) with substances that are reactive toward the functional groups of HB and which, besides at least one group that is reactive toward these functional groups of HB, comprise further groups such as carboxylate, sulfonate, diol.

The present invention also provides polymers obtainable by functionalization of the polymerized-in compound c) with substances that are reactive toward the functional groups of HB and which, besides at least one group that is reactive toward these functional groups of HB, comprise sugar molecules.

The present invention also provides polymers obtainable by functionalization of the polymerized-in compound c) with substances that are reactive toward the functional groups of HB and which, as well as at least one group that is reactive toward these functional groups of HB, comprise polar polymer chains such as, for example, polyacrylic acid chains.

The present invention also provides polymers obtainable by functionalization of the polymerized-in compound c) with substances that are reactive toward the functional groups of HB and which, as well as at least one group that is reactive toward these functional groups of HB, comprise nonpolar polymer chains such as, for example, polyisobutene chains.

The present invention also provides polymers obtainable by functionalization of the polymerized-in compound c) with substances that are reactive toward the functional groups of HB and which, as well as at least one group that is reactive toward these functional groups of HB, comprise silicone chains.

The present invention also provides polymers obtainable by functionalization of the polymerized-in compound c) with substances that are reactive toward the functional groups of HB and which, as well as at least one group that is reactive toward these functional groups of HB, comprise amphiphilic surfactant chains.

If the polymers according to the invention comprise groups that are reactive toward —NCO, modified polymers MP1 are also obtainable by

I) reaction of at least some of the groups that are reactive toward —NCO with a polyisocyanate, preferably with a diisocyanate,
II) reaction of the remaining NCO groups of the polyisocyanate with substances that are reactive toward NCO groups such as, for example, substances comprising hydroxyl groups or amine groups.

Also in accordance with the invention is thus a modified polymer MP1, where the compounds that are reactive toward the functional groups of the polymer P comprise isocyanate groups. These compounds that are reactive toward the functional groups of the polymer P are preferably polyisocyanates.

The aforementioned groups such as carboxylate, sulfonate, diol, sugars, polar and nonpolar polymer chains, surfactant chains can then preferably be bonded via a hydroxyl group or an amino group to the polymerized-in, NCO-functionalized hyperbranched polymer HB.

Also in accordance with the invention is a modified polymer MP2 obtainable by reacting a polymer MP1, where MP2 comprises, following the further reaction of MP1, structures selected from carboxylate, sulfonate, diol, sugars, polar polymer chains, nonpolar PIB chains, silicone chains and amphiphilic surfactant chains.

An embodiment of the present invention comprises modified polymers MP1 obtainable by functionalization of the polymerized-in compound c) with substances that are reactive toward the functional groups of HB, where in the range from 50 to 100 mol % of the functional groups of the hyperbranched polymer remaining after the polymerization are reacted with groups that are reactive toward these groups.

An embodiment of the present invention comprises modified polymers MP1 obtainable by functionalization of the polymerized-in compound c) with substances that are reative toward the functional groups of HB, where in the range from 50 to 75 mol % of the functional groups of the hyperbranched polymer remaining after the polymerization are reacted with groups that are reactive toward these groups.

An embodiment of the present invention is also the use of the polymers according to the invention for producing aqueous preparations. Preference is given here to preparations which comprise at least 5% by weight, in particular at least 20% by weight, very particularly preferably at least 30% by weight and most preferably at least 70% by weight, of water.

Preference is given to preparations which comprise at most 95% by weight, particularly preferably at most 90% by weight and especially at most 85% by weight, of water.

The preparations comprising water may be, for example, solutions, emulsions, suspensions or dispersions.

In addition to the polymers obtainable by the process according to the invention, further substances can be used for producing the preparations, such as e.g. customary auxiliaries (for example dispersants and/or stabilizers), surfactants, preservatives, antifoams, fragrances, wetting agents, UV filters, pigments, emollients, active ingredients, further thickeners, dyes, softeners, humectants and/or other polymers.

### Cosmetic Preparations

The invention further provides cosmetic preparations comprising at least one polymer according to the invention.

For the use in cosmetic preparations, preference is given to those polymers according to the invention which are prepared without using a catalyst comprising tin.

One advantage of the polymers according to the invention when they are used in cosmetic preparations is that their thickening power is in each case virtually unchanged even
1) after the addition of salts or pigments of more than 1% by weight, based on the preparation
2) up to temperatures of about 50° C. and
3) in the event of changes in the pH in the range from 2 to 13.

Cosmetic preparations which comprise the polymers according to the invention have a more finely divided structure compared to preparations which comprise known thickeners, as a result of the reduction in particle sizes.

The free functional groups which originate from the hyperbranched polymer HB bring about greater solubility in water, an increasing, in particular hydrophobic, degree of modification of the functional groups leads to an increasing thickening power. Likewise, by varying the modification, the rheological behavior can be adapted if necessary.

An embodiment of the present invention is the use of polymer-analogously polar modified polymers according to the invention for increasing the compatibility with polar solvents such as, for example, ethanol, propylene glycol or glycerol.

An embodiment of the present invention is the use of polymer-analogously polar modified polymers according to the invention for increasing the solubility of ingredients with limited solubility in water such as, for example, hydrophilic UV filters.

An embodiment of the present invention is the use of the polymer-analogously polar modified polymers according to the invention for increasing the water binding capacity in the preparation and also following application to the skin (moisturizer).

The use of the polymer-analogously nonpolar modified polymers according to the invention preferably leads to more stable emulsions, to increased compatibility with cosmetic oils and to a better skin feel.

An embodiment of the present invention is the use of the polymer-analogously nonpolar modified polymers according to the invention for increasing the compatibility with nonpolar liquid phases such as, for example, cosmetic oils—primarily also increased compatibility with silicone oils.

An embodiment of the present invention is the use of polymer-analogously nonpolar modified polymers according to the invention for increasing the solubility of ingredients of limited solubility in oil such as, for example, hydrophobic UV filters.

An embodiment of the present invention is the use of the polymer-analogously modified polymers according to the invention for improving the dispersibility of particles in the preparation.

An embodiment of the present invention is a method for improving the skin feel, characterized in that the skin is brought into contact with a preparation comprising a polymer-analogously nonpolar modified polymer according to the invention.

By using polymer-analogously (subsequently) amphiphilically modified polymers according to the invention, it is possible to adapt the rheological behavior as necessary.

The polymers according to the invention can generally be used instead of the associative thickeners known from the prior art for cosmetic preparations.

Cosmetic preparations comprising an associative thickener based on polyurethane are described in detail in WO 2009/135857, p. 22 to 73.

Preparations according to the invention are the preparations described in WO 2009/135857, p. 87 to 114, with the proviso that the preparations according to the invention comprise at least one polymer according to this invention instead of the polyurethane thickeners referred to therein.

Also in accordance with the invention are all preparations described in the publication IPCOM000181520D, with the proviso that the “polymer 1” specified therein is replaced by at least one polymer according to the present invention.

Also in accordance with the invention are all preparations described in the publication IPCOM000181842D, with the proviso that the “polymer 1” specified therein is replaced by at least one polymer according to the present invention.

Also in accordance with the invention are all preparations described in the publication IPCOM000183957D, with the proviso that the “polymer 1” specified therein is replaced by at least one polymer according to the present invention.

### EXAMPLES

The following examples illustrate the invention without limiting it thereto.

Synthesis examples of HB polymer cores

### Abbreviations Used:

TMP×3.2 EO: reaction product of trimethylolpropane with 3.2 molar excess of ethylene oxide.
TMP×12.2 PO: reaction product of trimethylolpropane with 12.2 molar excess of ethylene oxide.
TMP×15.7 PO: reaction product of trimethylolpropane with 15.7 molar excess of propylene oxide.

Unless described otherwise, percentages are percentages by weight.

Basonat® HI 100 (BASF SE): Polyisocyanurate based on hexamethylene diisocyanate, NCO content in accordance with DIN EN ISO 11909 21.5% by weight, viscosity at 23° C. in accordance with DIN EN ISO 3219 3500 mPas.

The hyperbranched polymers were analyzed by gel permeation chromatography using a refractometer as detector. The mobile phase used was dimethylacetamide (DMAc), tetrahydrofuran (THF) or hexafluoroisopropanol (HFIP), and the standard used for determining the molecular weight was polymethyl methacrylate (PMMA). The OH number was determined in accordance with DIN 53240, Part 2. The amine number was determined in accordance with DIN EN 13717.

### Synthesis Example 1

### Preparation of a Polar Hyperbranched Polycarbonate (HB.1)

In a 400 liter stirred-tank reactor with anchor stirrer, internal thermometer and distillation column, 200 kg of the trifunctional alcohol TMP×12.2 EO, 35.26 kg of diethyl carbonate and 0.04 kg of catalyst KOH were introduced as initial charge. The reaction mixture was heated to boiling with stirring and stirred until the boiling temperature of the reaction mixture had dropped to a temperature of 122° C. as a result of the evaporative cooling of the ethanol being released. Ethanol was then distilled off via the column and the temperature of the reaction mixture was slowly increased to 190° C. After an amount of 28.70 kg of distillate had been distilled off, the reaction mixture was cooled to 100° C. and stopped by adding 0.07 kg of 85% strength phosphoric acid. Then, remaining volatile constituents were removed at 140° C. and a pressure of 100 mbar over 120 min, and the mixture was then cooled to room temperature.

The hyperbranched polycarbonate was obtained in the form of a pale yellow resin (GPC (DMAc): Mn=3440 g/mol, Mw=6370 g/mol; OH number: 134 mg KOH/g polymer; viscosity (25° C.): 1600 mPas).

### Synthesis Example 2

### Preparation of a Weakly Polar Hyperbranched Polycarbonate (HB.2)

In a 40 liter stirred-tank reactor with anchor stirrer, internal thermometer and distillation column, 520.87 kg of the trifunctional alcohol TMP×3.2 EO, 9.12 kg of diethyl carbonate and 0.015 g of catalyst KOH were introduced as initial charge. The reaction mixture was heated to boiling with stirring and stirred until the boiling temperature of the reaction mixture had dropped to a temperature of 118° C. as a result of the evaporative cooling of the ethanol being released. Then, ethanol was distilled off via the column and the temperature of the reaction mixture was slowly increased to 190° C. After an amount of 5.80 kg of distillate had been distilled off, the reaction mixture was cooled to 140° C. and stopped by adding 0.025 kg of 85% strength phosphoric acid. Then, remaining volatile constituents were removed at 140° C. and a pressure of 100 mbar over 3 h, and the mixture was then cooled to room temperature.

The hyperbranched polycarbonate was obtained in the form of a pale yellow resin (GPC (DMAc): Mn=1740 g/mol, Mw=5020 g/mol; OH number: 256 mg KOH/g polymer).

### Synthesis Example 3

### Preparation of a Nonpolar Hyperbranched Polycarbonate (HB.3)

In a 400 liter stirred-tank reactor with anchor stirrer, internal thermometer and distillation column, 257.82 kg of the trifunctional alcohol TMP×15.7 PO and 32.18 kg of diethyl carbonate were introduced as initial charge and admixed with a solution of 0.174 kg KOH in 1.164 kg of ethanol. The reaction mixture was heated to boiling and stirred until the boiling temperature of the reaction mixture had dropped to a temperature of 139° C. as a result of the evaporative cooling of the ethanol being released. Then, ethanol was distilled off via the column and the temperature of the reaction mixture was slowly increased to 200° C. After an amount of 18.0 kg of distillate had been distilled off, the reaction mixture was cooled to 140° C. and stopped by adding 0.358 kg of 85% strength phosphoric acid. Then, remaining volatile constituents were removed at 140° C. and a pressure of 100 mbar over 3 h, and the mixture was then cooled to room temperature.

The hyperbranched polycarbonate was obtained in the form of a pale yellow resin (GPC (THF): Mn=2920 g/mol, Mw=5570 g/mol; OH number: 91 mg KOH/g polymer; viscosity (25° C.): 650 mPas).

### Synthesis Example 4

### Preparation of a Hyperbranched Polyether Amine Polyol (HB.4)

In a four-neck flask, equipped with stirrer, distillation bridge, gas inlet tube and internal thermometer, 2000 g of triethanolamine and 13.4 g of 50% strength aqueous hypophosphorous acid were introduced as initial charge and the mixture was slowly heated to 230° C., during which, at about 220° C., the formation of condensate started. The reaction mixture was then stirred for 5 h at 230° C., during which, the condensate forming during the reaction was removed by means of a moderate stream of nitrogen as stripping gas via the distillation bridge. After 5 h had passed, the mixture was cooled to 140° C. and the pressure was reduced slowly and stepwise to 50 mbar in order to remove any remaining volatile fractions.

The product mixture was then cooled to room temperature.

The product had the following characteristic data:

Mn=4900 Da, Mw=14700 Da. (GPC (HFIP))

OH number=460 mg KOH/g

### Synthesis Example 5

### Preparation of a Hyperbranched Polyisocyanurate (HB.5)

In a 4 liter glass flask, equipped with stirrer, internal thermometer and distillation unit, 1045.2 g of tris(hydroxyethyl) isocyanurate (THEIC), 424.2 g of diethylene glycol, 300 g of water and 3 g of sulfuric acid (95% strength by weight) were introduced as initial charge, heated to 90° C. and stirred for 1 h at standard pressure. Then, the internal temperature was slowly increased to 170° C., the mixture was stirred for 10 h, and the distillate passing over was collected. Then, the reaction mixture was cooled to 120° C., neutralized with 50% strength aqueous NaOH solution, poured into an aluminum dish and cooled.

The product had the following characteristic data:

Mn=2200 Da, Mw=63500 Da (GPC (DMAc))

OH number: 243 mg KOH/g

### Synthesis Example 6

### Preparation of a Hyperbranched Polyurea (HB.6)

In a reaction vessel which was equipped with stirrer, internal thermometer, reflux condenser and nitrogen inlet tube, with gassing with dry nitrogen, 646.5 g of Basonat® HI 100 were introduced as initial charge and heated to 80° C. with stirring. Then, with continuous stirring over a period of 2 h, 498.0 g of n-butanol were added such that the temperature of the reaction mixture does not exceed 80° C. When the addition was complete, the reaction mixture was stirred for a further 3 h at 80° C.

The mixture was then cooled to 50° C., the reflux condenser was exchanged for a descending condenser with capture vessel, and the reaction mixture was admixed with 355.5 g of isophoronediamine and 0.1 g of dibutyltin dilaurate. The reaction mixture was then heated to 170° C. with stirring and stirred for 5 h at this temperature, during which n-butanol being released during the reaction was separated off by distillation and collected. During this time, the amine consumption in the reaction mixture was monitored by means of titration with 0.1N HCl and, in so doing, the conversion was ascertained as a percentage of the theoretically possible complete conversion. After reaching an amine conversion of 42 mol % (i.e. 58 mol % remaining amine), the reaction was terminated by cooling to room temperature.

The amount of butanol in the distillate was 249 g.

The product had the following characteristic data:

Mn=2500 Da, Mw=5200 Da. (GPC (HFIP))

Amine number=0.5 g of primary amine/100 g of polymer, calculated with mass of nitrogen=14.007 g/mol.

### Synthesis Examples of Functional PUR Associative Thickeners

### General Remarks:

The molecular weights of the thickeners A.1-A.12 were determined by GPC in THF (tetrahydrofuran) as solvent, standard: PMMA.

All of the reactions were carried out under a protective-gas atmosphere (dried nitrogen)

Unless expressly stated otherwise, data in % are always % by weight.

### Synthesis Example V1

### Preparation of a PUR Associative Thickener Comprising a Hyperbranched Polyisocyanurate, Degree of Functionalization of the OH Groups 50% (A.1)

120.00 g of polyethylene glycol Pluriol®E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 110 ppm. The polymer solution was then cooled to 50° C. (internal temperature) and admixed with 89 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.40%. Then, 16.58 g of Lutensol® AT11 (BASF SE), dissolved in 20 ml of xylene, were added and the reaction mixture was further heated at 50° C. until the isocyanate content was 0.16%. Then, 5.35 g of the hyperbranched polyisocyanurate HB.5, dissolved in 20 ml of THF, were added and the reaction mixture was further heated at 50° C. until the isocyanate content was finally 0%. The solvents xylene and THF were then largely removed by vacuum distillation at elevated temperature (ca. 60° C.) (residual content<100 ppm) and the residue was dissolved in 599.9 g of water. 7.49 g of the preservative Euxyl® K701 and 80 mg of the stabilizer 4-hydroxy-TEMPO were then added to the aqueous solution. After cooling to room temperature (25° C.), the polymer A.1 (Mₙ=14 000 g/mol; M_{w}=36 400 g/mol) was obtained in the form of an aqueous dispersion which had a solids content of 20.5%. The viscosity of a 10% strength aqueous solution of the branched, functional polyurethane A.1 was 33 000 mPa*s (shear rate 100 1/s) (viscosity cannot be measured at shear rate 350 1/s).

### Synthesis Example V2

### Preparation of a PUR Associative Thickener Comprising a Polar Hyperbranched Polycarbonate, Degree of Functionalization of the OH Groups 50% (A.2)

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 100 ppm. The polymer solution was then cooled to 50° C. (internal temperature) and admixed with 89 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.40%. Then, 16.58 g of Lutensol® AT11 (BASF SE), dissolved in 20 ml of xylene, were added and the reaction mixture was further heated at 50° C. until the isocyanate content was 0.17%. Then, 14.74 g of the polar, hyperbranched polycarbonate HB.1, dissolved in 20 ml of xylene, were added and the reaction mixture was further heated at 50° C. until the isocyanate content was finally 0%. The solvent xylene was then largely removed by vacuum distillation at elevated temperature (ca. 60° C.) (residual content<100 ppm) and the residue was dissolved in 646.9 g of water. 8.05 g of the preservative Euxyl® K701 and 80 mg of the stabilizer 4-hydroxy-TEMPO were then added to the aqueous solution. After cooling to room temperature (25° C.), the polymer A.2 (Mₙ=13 900 g/mol; M_{w}=38 800 g/mol) was obtained in the form of an aqueous dispersion which had a solids content of 20.5%. The viscosity of a 10% strength aqueous solution of the branched, functional polyurethane A.2 was 27 000 mPa*s (shear rate 100 1/s) (viscosity cannot be measured at shear rate 350 1/s).

### Synthesis Example V3

### Preparation of a PUR Associative Thickener Comprising a Weakly Polar Hyperbranched Polycarbonate, Degree of Functionalization of the OH Groups 50% (A.3)

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 100 ppm. Then, the polymer solution was cooled to 50° C. (internal temperature) and admixed with 89 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.40%. Then, 16.58 g of Lutensol® AT11 (BASF SE), dissolved in 20 ml of xylene, were added and the reaction mixture was further heated at 50° C. until the isocyanate content was 0.17%. Then, 7.72 g of the weakly polar, hyperbranched polycarbonate HB.2, dissolved in 20 ml of xylene, were added and the reaction mixture was further heated at 50° C. until the isocyanate content was finally 0%. The solvent xylene was then largely removed by vacuum distillation at elevated temperature (ca. 60° C.) (residual content<100 ppm) and the residue was dissolved in 611.8 g of water. Then, 7.63 g of the preservative Euxyl® K701 and 80 mg of the stabilizer 4-hydroxy-TEMPO were added to the aqueous solution. After cooling to room temperature (25° C.), the polymer A.3 (Mₙ=16 000 g/mol; M_{w}=40 600 g/mol) was obtained in the form of an aqueous dispersion which had a solids content of 20.7%. The viscosity of a 10% strength aqueous solution of the branched, functional polyurethane A.3 was 34 000 mPa*s (shear rate 100 1/s) (viscosity cannot be measured at shear rate 350 1/s).

### Synthesis Example V4

### Preparation of a PUR Associative Thickener Comprising a Nonpolar Hyperbranched Polycarbonate, Degree of Functionalization of the OH Groups 50% (A.4)

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 90 ppm. Then, the polymer solution was cooled to 50° C. (internal temperature) and admixed with 89 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.40%. Then, 16.58 g of Lutensol® AT11 (BASF SE), dissolved in 20 ml of xylene, were added and the reaction mixture was further heated at 50° C. until the isocyanate content was 0.17%. Then, 21.70 g of the nonpolar, hyperbranched polycarbonate HB.3, dissolved in 20 ml of xylene, were added and the reaction mixture was further heated at 50° C. until the isocyanate content was finally 0%. The solvent xylene was then largely removed by vacuum distillation at elevated temperature (ca. 60° C.) (residual content<100 ppm) and the residue was dissolved in 681.7 g of water. Then, 8.47 g of the preservative Euxyl® K701 and 90 mg of the stabilizer 4-hydroxy-TEMPO were added to the aqueous solution. After cooling to room temperature (25° C.), the polymer A.4 (Mₙ=12 200 g/mol; M_{w}=33 200 g/mol) was obtained in the form of an aqueous dispersion which had a solids content of 19.7%. The viscosity of a 10% strength aqueous solution of the branched, functional polyurethane A.4 was 38 000 mPa*s (shear rate 100 1/s) (viscosity cannot be measured at shear rate 350 1/s).

### Synthesis Example V5

### Preparation of a PUR Associative Thickener Comprising a Hyperbranched Polyurea, Degree of Functionalization of the OH Groups Ca. 50% (A.5)

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 100 ppm. Then, the polymer solution was cooled to 50° C. (internal temperature) and admixed with 89 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.41%. Then, 16.58 g of Lutensol® AT11 (BASF SE), dissolved in 20 ml of xylene, were added and the reaction mixture was further heated at 50° C. until the isocyanate content was 0.18%. Then, 16.46 g of the hyperbranched polyurea HB.6, dissolved in 50 ml of THF, were added and the reaction mixture was further heated at 50° C. until the isocyanate content was finally 0%. The solvents xylene and THF were then largely removed by vacuum distillation at elevated temperature (ca. 60° C.) (residual content<100 ppm) and the residue was dissolved in 639.0 g of water. Then, 7.99 g of the preservative Euxyl® K701 and 80 mg of the stabilizer 4-hydroxy-TEMPO were added to the aqueous solution. After cooling to room temperature (25° C.), the polymer A.5 (Mₙ=11 600 g/mol; M_{w}=28 600 g/mol) was obtained in the form of an aqueous dispersion which had a solids content of 20.4%. The viscosity of a 10% strength aqueous solution of the branched, functional polyurethane A.5 was 17 000 mPa*s (shear rate 100 1/s) (viscosity cannot be meassured at shear rate 3501/s).

### Synthesis Example V6

### Preparation of a PUR Associative Thickener Comprising a Hyperbranched Polyurea, Degree of Functionalization of the OH Groups 100% (A.6)

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 90 ppm. Then, the polymer solution was cooled to 50° C. (internal temperature) and admixed with 89 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.41%. Then, 16.58 g of Lutensol® AT11 (BASF SE), dissolved in 20 ml of xylene, were added and the reaction mixture was further heated at 50° C. until the isocyanate content was 0.15%. Then, 8.59 g of the hyperbranched polyurea HB.6, dissolved in 20 ml of THF, were added and the reaction mixture was further heated at 50° C. until the isocyanate content was finally 0%. The solvent xylene and THF were then largely removed by vacuum distillation at elevated temperature (ca. 60° C.) (residual content<100 ppm) and the residue was dissolved in 607.5 g of water. Then, 7.60 g of the preservative Euxyl® K701 and 80 mg of the stabilizer 4-hydroxy-TEMPO were added to the aqueous solution. After cooling to room temperature (25° C.), the polymer A.6 (Mₙ=13 700 g/mol; M_{w}=34 000 g/mol) was obtained in the form of an aqueous dispersion which had a solids content of 20.1%. The viscosity of a 10% strength aqueous solution of the branched, functional polyurethane A.6 was 47 000 mPa*s (shear rate 100 1/s) (viscosity cannot be measured at shear rate 350 1/s).

### Synthesis Example V7

### Preparation of a PUR Associative Thickener Comprising a Hyperbranched Polyurea, Degree of Functionalization of the OH Groups 100% (A.7)

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 100 ppm. Then, the polymer solution was cooled to 50° C. (internal temperature) and admixed with 89 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 8.89 g of isophorone diisocyanate, dissolved in 10 ml of xylene, the polymerization was started and the batch was run at an internal temperature of 50° C. to an isocyanate content of 0.40%. Then, a mixture of 8.29 g of Lutensol® AT11 (BASF SE) and 7.17 g of Lutensol® TO10 (BASF SE), dissolved in 20 ml of xylene, were added and the reaction mixture was further heated at 50° C. until the isocyanate content was 0.16%. Then, 8.59 g of the hyperbranched polyurea HB.6, dissolved in 20 ml of THF, were added and the reaction mixture was further heated at 50° C. until the isocyanate content was finally 0%. The solvents xylene and THF were then largely removed by vacuum distillation at elevated temperature (ca. 60° C.) (residual content<100 ppm) and the residue was dissolved in 583.1 g of water. Then, 7.29 g of the preservative Euxyl® K701 and 70 mg of the stabilizer 4-hydroxy-TEMPO were added to the aqueous solution. After cooling to room temperature (25° C.), the polymer A.7 (Mₙ=12 500 g/mol; M_{w}=31 200 g/mol) was obtained in the form of an aqueous dispersion which had a solids content of 19.8%. The viscosity of a 10% strength aqueous solution of the branched, functional polyurethane A.7 was 22 000 mPa*s (shear rate 100 1/s) (viscosity cannot be measured at shear rate 350 1/s).

### Synthesis Example V8

### Preparation of a PUR Associative Thickener Comprising a Hyperbranched Polyether Amine Polyol, Degree of Functionalization of the OH Groups 50% (A.8)

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 90 ppm. Then, the polymer solution was cooled to 50° C. (internal temperature) and admixed with 89 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.41%. Then, a mixture of 8.29 g of Lutensol® AT11 (BASF SE) and 7.17 g of Lutensol® TO10 (BASF SE), dissolved in 20 ml of xylene, was added and the reaction mixture was further heated at 50° C. until the isocyanate content was 0.17%. Then, 4.51 g of the hyperbranched polyetherpolyol HB.4, dissolved in 20 ml of THF, were added and the reaction mixture was further heated at 50° C. until the isocyanate content was finally 0%. The solvents xylene and THF were then largely removed by vacuum distillation at elevated temperature (ca. 60° C.) (residual content<100 ppm) and the residue was dissolved in 555.4 g of water. Then, 6.95 g of the preservative Euxyl® K701 and 70 mg of the stabilizer 4-hydroxy-TEMPO were added to the aqueous solution. After cooling to room temperature (25° C.), the polymer A.8 (Mₙ=8700 g/mol; M_{w}=19 800 g/mol) was obtained in the form of an aqueous dispersion which had a solids content of 21.2%. The viscosity of a 10% strength aqueous solution of the branched, functional polyurethane A.8 was 4000 mPa*s (shear rate 100 1/s) and 2700 mPa*s (shear rate 350 1/s).

### Synthesis Example V9

### Preparation of a PUR Associative Thickener Based on a Polar Hyperbranched Polycarbonate, Degree of Functionalization of the OH Groups 100% (A.9)

415.80 g of Lutensol® AT80 (BASF SE) were dissolved in 415.80 g of acetone under nitrogen in a 2 l polymerization reactor (flat flange glass vessel with anchor stirrer). Then, the polymer solution was heated to 50° C. (internal temperature) and admixed with 403 mg of acetic acid in order to neutralize the amount of potassium acetate in the Lutensol® quantitatively determined beforehand. By adding 4 mg of zinc neodecanoate (TIB Kat 616, TIB Chemicals, Mannheim) and 22.23 g of isophorone diisocyanate, dissolved in 22.23 g of acetone, the reaction was started and the batch was run at an internal temperature of 50° C. to an isocyanate content of 0.40%. Then, 41.87 g of the polar, hyperbranched polycarbonate HB.1, dissolved in 41.87 g of acetone, and also a further 1.44 g of zinc neodecanoate (TIB Kat 616, TIB Chemicals, Mannheim), dissolved in 10.00 g of acetone, were added and the reaction mixture was further heated at 50° C. until the isocyanate content was finally 0%. The solvent acetone was then largely removed by vacuum distillation at elevated temperature (ca. 60° C.) (residual content<100 ppm). After cooling to room temperature (25° C.), the polymer A.9 (Mₙ=5300 g/mol; M_{w}=7200 g/mol) was obtained in the form of a highly viscous liquid. The viscosity of a 10% strength aqueous solution of the branched, functional polyurethane A.9 was 2650 mPa*s (shear rate 100 1/s) and 2550 mPa*s (shear rate 350 1/s).

### Synthesis Example V10

### Preparation of a PUR Associative Thickener Based on a Polar Hyperbranched Polycarbonate, Degree of Functionalization of the OH Groups 100% (A.10)

415.80 g of Lutensol® AT80 (BASF SE) were dissolved in 415.80 g of acetone under nitrogen in a 2 l polymerization reactor (flat flange glass vessel with anchor stirrer). Then, the polymer solution was heated to 50° C. (internal temperature) and admixed with 403 mg of acetic acid in order to neutralize the amount of potassium acetate in the Lutensol® quantitatively determined beforehand. By adding 4 mg of zinc neodecanoate (TIB Kat 616, TIB Chemicals, Mannheim) and 16.80 g of hexamethylene diisocyanate, dissolved in 16.80 g of acetone, the reaction was started and the batch was run at an internal temperature of 50° C. to an isocyanate content of 0.49%. Then, 41.87 g of the polar, hyperbranched polycarbonate HB.1, dissolved in 41.87 g of acetone, and also a further 1.42 g of zinc neodecanoate (TIB Kat 616, TIB Chemicals, Mannheim), dissolved in 10.00 g of acetone, were added and the reaction mixture was further heated at 50° C. until the isocyanate content was finally 0%. The solvent acetone was then largely removed by vacuum distillation at elevated temperature (ca. 60° C.) (residual content<100 ppm). After cooling to room temperature (25° C.), the polymer A.10 (Mₙ=5800 g/mol; M_{w}=8500 g/mol) was obtained in the form of a highly viscous liquid. The viscosity of a 10% strength aqueous solution of the branched, functional polyurethane A.10 was 14 000 mPa*s (shear rate 100 1/s) and 9500 mPa*s (shear rate 350 1/s).

### Comparison

### Synthesis Example V11

### Preparation of a PUR Associative Thickener Comprising Trimethylolpropane (Branched Structure Comparable with the Prior Art), Degree of Functionalization of the OH Groups 100% (A.11)

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 120 ppm. Then, the polymer solution was cooled to 50° C. (internal temperature) and admixed with 89 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.40%. Then, 16.58 g of Lutensol® AT11 (BASF SE), dissolved in 20 ml of xylene, were added and the reaction mixture was further heated at 50° C. until the isocyanate content was 0.18%. Then, 0.79 g of 1,1,1-tris(hydroxymethyl)propane (TMP), dissolved in 20 ml of THF, were added and the reaction mixture was further heated at 50° C. until the isocyanate content was finally 0%. The solvents xylene and THF were then largely removed by vacuum distillation at elevated temperature (ca. 60° C.) (residual content<100 ppm) and the residue was dissolved in 577.1 g of water. Then, 7.22 g of the preservative Euxyl® K701 and 70 mg of the stabilizer 4-hydroxy-TEMPO were added to the aqueous solution. After cooling to room temperature (25° C.), the polymer A.11 (Mₙ=16 500 g/mol; M_{w}=39 500 g/mol) was obtained in the form of an aqueous dispersion which had a solids content of 20.5%. The viscosity of a 5% strength aqueous solution of the branched polyetherpolyurethane A.11 was 12 500 mPa*s (shear rate 100 1/s) and 7500 mPa*s (shear rate 350 1/s).

### Comparison

### Synthesis Example V12

### Preparation of a PUR Associative Thickener Comprising Ethylene Glycol (Linear Structure), Degree of Functionalization of the OH Groups 100% (A.12)

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 100 ppm. Then, the polymer solution was cooled to 50° C. (internal temperature) and admixed with 89 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.40%. Then, 16.58 g of Lutensol® AT11 (BASF SE), dissolved in 20 ml of xylene, were added and the reaction mixture was further heated at 50° C. until the isocyanate content was 0.18%. Then, 0.55 g of monoethylene glycol, dissolved in 20 ml of THF, were added and the reaction mixture was further heated at 50° C. until the isocyanate content was finally 0%. The solvents xylene and THF were largely removed by vacuum distillation at elevated temperature (ca. 60° C.) (residual content<100 ppm) and the residue was dissolved in 575.9 g of water. Then, 7.20 g of the preservative Euxyl® K701 and 70 mg of the stabilizer 4-hydroxy-TEMPO were added to the aqueous solution. After cooling to room temperature (25° C.), the polymer A.12 (Mₙ=14300 g/mol; M_{w}=33500 g/mol) was obtained in the form of an aqueous dispersion which had a solids content of 19.9%. The viscosity of a 10% strength aqueous solution of the branched polyetherpolyurethane A.12 was 27 000 mPa*s (shear rate 100 1/s) (viscosity cannot be measured at shear rate 350 1/s).

### Synthesis Examples for Modified Polymers MP1 and MP2

### Synthesis Example MP2.1

### Preparation of a PUR Associative Thickener Comprising a Nonpolar Hyperbranched Polycarbonate, Degree of Functionalization of the OH Groups 50% and Post-Functionalization with Diisocyanates and Alkyl Chains

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 120 ppm. Then, the polymer solution was cooled to 50° C. (internal temperature) and admixed with 59 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.41%. Then, a mixture of 8.29 g of Lutensol® AT11 (BASF SE) and 7.17 g of Lutensol® TO10 (BASF SE), dissolved in 20 ml of xylene, was added and the reaction mixture was further heated at 50° C. until the isocyanate content was 0.15%. Then, 21.70 g of the nonpolar, hyperbranched polycarbonate HB.3, dissolved in 20 ml of xylene, were added and the reaction mixture was further heated at 50° C. until the isocyanate content was finally 0%. Subsequently, 3.91 g of isophorone diisocyanate, dissolved in 10 ml of xylene, were added, and the batch was run to an isocyanate content of 0.15%. To the polymer MP1.1 thus obtained 4.96 g of octadecanol were then added and the mixture was further heated at 50° C. until the isocyanate content was 0%. The solvent xylene was then largely removed by vacuum distillation at elevated temperature (ca. 60° C.) (residual content<100 ppm) and the residue was dissolved in 711.9 g of water. Then, 8.85 g of the preservative Euxyl® K701 and 90 mg of the stabilizer 4-hydroxy-TEMPO were added to the aqueous solution. After cooling to room temperature (25° C.), the polymer MP2.1 (Mₙ=10 400 g/mol; M_{w}=24 500 g/mol) was obtained in the form of an aqueous dispersion which had a solids content of 19.7%. The viscosity of a 10% strength aqueous solution of the branched, modified polyurethane MP2.1 was 10 800 mPa*s (shear rate 100 1/s) and 6200 mPa*s (shear rate 350 1/s).

### Synthesis Example MP2.2

### Preparation of a PUR Associative Thickener Comprising a Nonpolar Hyperbranched Polycarbonate, Degree of Functionalization of the OH Groups 50% and Post-Functionalization with Diisocyanates and a Silicone Chains

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 110 ppm. Then, the polymer solution was cooled to 50° C. (internal temperature) and admixed with 59 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.41%. Then, a mixture of 8.29 g of Lutensol® AT11 (BASF SE) and 7.17 g of Lutensol® TO10 (BASF SE), dissolved in 20 ml of xylene, was added and the reaction mixture was further heated at 50° C. until the isocyanate content was 0.18%. Then, 21.70 g of the nonpolar, hyperbranched polycarbonate HB.3, dissolved in 20 ml of xylene, were added and the reaction mixture was further heated at 50° C. until the isocyanate content was finally 0%. Subsequently, 3.91 g of isophorone diisocyanate, dissolved in 10 ml of xylene, were added, and the batch was run to an isocyanate content of 0.15%. To the polymer MP1.2 thus obtained 44 g tegomer H—Si 2311 (molecular weight 2500 g/mol) were then added and the mixture was further heated at 50° C. until the isocyanate content was 0%. The solvent xylene was then largely removed by vacuum distillation at elevated temperature (ca. 60° C.) (residual content<100 ppm) and the residue was dissolved in 868.9 g of water. Then, 10.81 g of the preservative Euxyl® K701 and 110 mg of the stabilizer 4-hydroxy-TEMPO were added to the aqueous solution. After cooling to room temperature (25° C.), the polymer MP2.2 (Mₙ=12 100 g/mol; M_{w}=27 800 g/mol) was obtained in the form of an aqueous dispersion which had a solids content of 19.9%. The viscosity of a 10% strength aqueous solution of the branched, modified polyurethane MP2.2 was 10 000 mPa*s (shear rate 100 1/s) and 5600 mPa*s (shear rate 350 1/s).

### Synthesis Example MP2.3

### Preparation of a PUR Associative Thickener Comprising a Nonpolar Hyperbranched Polycarbonate, Degree of Functionalization of the OH Groups 50% and Post-Functionalization with Diisocyanates and Dialkylamines

120.00 g of polyethylene glycol Pluriol® E6000 (BASF SE, molecular weight 6000 g/mol) were dissolved in 467.00 g of xylene under nitrogen in a 2 l polymerization reactor (flat flange glass vessel with anchor stirrer). After heating the solution to ca. 140° C. (internal temperature), exactly 200 g of xylene were distilled off. The water content of the reaction mixture was then only still ca. 100 ppm. Then, the polymer solution was cooled to 50° C. (internal temperature) and admixed with 59 mg of acetic acid, dissolved in 5 ml of xylene, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of xylene, and 6.72 g of hexamethylene diisocyanate, dissolved in 10 ml of xylene, the polymerization was started and the batch was run at an internal temperature of 50° C. to an isocyanate content of 0.41%. Then, a mixture of 8.29 g of Lutensol® AT11 (BASF SE) and 7.17 g of Lutensol® TO10 (BASF SE), dissolved in 20 ml of xylene, was added and the reaction mixture was further heated at 50° C. until the isocyanate content was 0.18%. Then, 21.70 g of the nonpolar, hyperbranched polycarbonate HB.3, dissolved in 20 ml of xylene, were added and the reaction mixture was further heated at 50° C. until the isocyanate content was finally 0%. Subsequently, 3.91 g of isophorone diisocyanate, dissolved in 10 ml of xylene, were added, and the batch was run to an isocyanate content of 0.16%. To the polymer MP1.3 thus obtained 6.72 g ditridecylamine were then added and the mixture was further heated at 50° C. until the isocyanate content was 0%. The solvent xylene was then largely removed by vacuum distillation at elevated temperature (ca. 60° C.) (residual content<100 ppm) and the residue was dissolved in 719.7 g of water. Then, 8.95 g of the preservative Euxyl® K701 and 90 mg of the stabilizer 4-hydroxy-TEMPO were added to the aqueous solution. After cooling to room temperature (25° C.), the polymer MP2.3 (Mₙ=11 000 g/mol; M_{w}=26 700 g/mol) was obtained in the form of an aqueous dispersion which had a solids content of 20.1%. The viscosity of a 10% strength aqueous solution of the branched, modified polyurethane MP2.3 was 8800 mPa*s (shear rate 100 1/s) and 5300 mPa*s (shear rate 350 1/s).

### Synthesis Example MP2.4

### Preparation of a PUR Associative Thickener Comprising a Nonpolar Hyperbranched Polycarbonate, Degree of Functionalization of the OH Groups 50% and Post-Functionalization with Diisocyanates and Amino Sugars

120.00 g of polyethylene glycol Pluriol® E6000 (BASF SE, molecular weight 6000 g/mol) were freed from traces of water at 120° C. in vacuo and were then dissolved in 267.00 g of acetone under nitrogen in a 2 l polymerization reactor (flat flange glass vessel with anchor stirrer). The water content of the reaction mixture was ca. 290 ppm. The polymer solution was admixed with 59 mg of acetic acid, dissolved in 5 ml of acetone, in order to neutralize the amount of potassium acetate in the polyethylene glycol quantitatively determined beforehand. By adding 360 mg of zinc neodecanoate (TIB Kat 616, TIB Chemicals, Mannheim), dissolved in 5 ml of acetone, and 6.72 g of hexamethylene diisocyanate, dissolved in 10 ml of acetone, the polymerization was started and the batch was run at an internal temperature of 50° C. to an isocyanate content of 0.42%. Then, a mixture of 8.29 g of Lutensol® AT11 (BASF SE) and 7.17 g of Lutensol® TO10 (BASF SE), dissolved in 20 ml of acetone, was added and the reaction mixture was further heated at 50° C. until the isocyanate content was 0.16%. Then, 21.70 g of the nonpolar, hyperbranched polycarbonate HB.3, dissolved in 20 ml of acetone, were added and the reaction mixture was further heated at 50° C. until the isocyanate content was finally 0%. Subsequently, 3.91 g of isophorone diisocyanate, dissolved in 10 ml of xylene, were added, and the batch was run to an isocyanate content of 0.16%. To the polymer MP1.4 thus obtained 4.68 g of the sugar amine 2,3,4,5,6-pentahydroxy-N-[3-(methylamino)propyl]hexamide, dissolved in 10 ml of water, were then added and the mixture was further heated at 50° C. until the isocyanate content was 0%. The solvent acetone was then largely removed by vacuum distillation at elevated temperature (ca. 60° C.) (residual content<100 ppm) and the residue was dissolved in 696.4 g of water. Then, 8.71 g of the preservative Euxyl® K701 and 90 mg of the stabilizer 4-hydroxy-TEMPO were added to the aqueous solution. After cooling to room temperature (25° C.), the polymer MP2.4 (Mₙ=7100 g/mol; M_{w}=14 700 g/mol) was obtained in the form of an aqueous dispersion which had a solids content of 19.6%. The viscosity of a 10% strength aqueous solution of the branched, modified polyurethane MP2.4 was 1400 mPa*s (shear rate 100 1/s) and 1200 mPa*s (shear rate 350 1/s).

### Formulation Examples

### Preparation of Cosmetic Formulations Using the PUR Associative Thickeners A.1-A.12; Cremophor® A6/Cremophor® A25 Served as Formulation Base (Examples FA.1.1-FA.1.12)

The cosmetic formulations were prepared by adding the water phase B to the oil phase A and subsequently admixing the resulting O/W emulsion with the preservative (phase C). This gave the formulations FA.1.1-FA.1.12 based on a Cremophor® A6/Cremophor® A25 base (Tab. 1 and Tab. 2) and also the formulations FA.2.1-FA.2.12 based on a stearate base (Tab. 3 and Tab. 4).

Quantitative data of the Examples A.1-A.12 in the formulations FA.1.1-FA.1.12 (Tab.1) and FA.2.1-FA.2.12 (Tab.3) give amounts of polymer.

TABLE 1
Formulation parameters for the cosmetic formulations FA.1.1-FA.1.12
based on a Cremophor ®A6/Cremophor ® A25 base.

| Phase | Ingredients | FA.1.1-1.12* |
|---|---|---|
| Phase A | Cremophor ® A 6 | 2.0 g |
| | Cremophor ® A 25 | 2.0 g |
| | Lanette ® O | 2.5 g |
| | Paraffin oil | 5.0 g |
| | Luvitol ® EHO | 5.0 g |
| Phase B | PUR thickener A.1-A.12 | 0.5 g |
| | 1,2-Propylene glycol | 5.0 g |
| | Water | 77.5 g |
| Phase C | Euxyl ® K300 | 0.5 g |

TABLE 2
Viscosities of the cosmetic formulations FA.1.1-FA.1.12
as a function of the salt concentration.

| | Viscosity [Pa * s] |
|---|---|
| Formulation | in the presence of 2.0% NaCl |
| FA.1.1 | 22.0 |
| FA.1.2 | 20.6 |
| FA.1.3 | 24.8 |
| FA.1.4 | 37.9 |
| FA.1.5 | 11.9 |
| FA.1.6 | 26.7 |
| FA.1.7 | 9.5 |
| FA.1.8 | 12.0 |
| FA.1.9 | 8.1 |
| FA.1.10 | 7.9 |
| FA.1.11* | 30.0 |
| FA.1.12* | 20.0 |

*not according to the invention
FA.1.11 and FA.1.12 exhibited very poor, gritty structure.

Furthermore, the viscosity in Pa*s of formulation Z 1.7 from WO 2009/135857 in the presence of 2.0% NaCl was determined for comparison. This was 9.1. This comparison shows that the thickeners according to the invention comprising a polymerized-in hyperbranched polymer HB can bring about a stronger increase in the viscosity compared with thickeners without polymerized-in hyperbranched polymer HB as disclosed in WO 2009/135857.

TABLE 3
Formulation parameters for the cosmetic formulations
FA.2.1-FA.2.12 based on a stearate base

| Phase | Ingredients | FA.2.1-FA.2.12 |
|---|---|---|
| Phase A | Cutina ® GMS | 2.0 g |
| | Lanette ® 18 | 2.0 g |
| | Dow Corning ® 345 Fluid | 3.0 g |
| | Cetiol ® OE | 3.0 g |
| | Abil ® 350 | 2.0 g |
| | Dry Flo PC | 1.0 g |
| | Myrj ® 52 | 2.0 g |
| Phase B | PUR thickener A.1 to A.12 | 0.5 g |
| | Glycerol | 5.0 g |
| | Water | 79.0 g |
| Phase C | Euxyl ® K300 | 0.5 g |

TABLE 4
Viscosities of the cosmetic formulations FA.2.1-FA.2.12
as a function of the salt concentration.

| | Viscosity [Pa * s] |
|---|---|
| Formulation | in the presence of 2.0% NaCl |
| FA.2.1 | 8.1 |
| FA.2.2 | 5.3 |
| FA.2.3 | 6.6 |
| FA.2.4 | 7.8 |
| FA.2.5 | 9.5 |
| FA.2.6 | 8.0 |
| FA.2.7 | 11.1 |
| FA.2.8 | 9.1 |
| FA.2.9 | 4.9 |
| FA.2.10 | 3.2 |
| FA.2.11* | 9.0 |
| FA.2.12* | 4.4 |

*not according to the invention

TABLE 5
Viscosities of the thickeners A.1-A.12 in water,
as a function of the shear rate.

| | |
|---|---|
| Polymer | Viscosity |
| concentration | [mPa * s] |

| | in water | shear rate 100 | shear rate 350 |
|---|---|---|---|
| Polymer | [% by weight] | 1/s | 1/s |
| A.1 | 10 | 33 000 | n.d. |
| A.2 | 10 | 27 000 | n.d. |
| A.3 | 10 | 34 000 | n.d. |
| A.4 | 10 | 38 000 | n.d. |
| A.5 | 10 | 17 000 | n.d. |
| A.6 | 10 | 47 000 | n.d. |
| A.7 | 10 | 22 000 | n.d. |
| A.8 | 10 | 4000 | 2700 |
| A.9 | 10 | 2650 | 2550 |
| A.10 | 10 | 14 000 | 9500 |
| A.11* | 5 | 12 500 | 7500 |
| A.12* | 10 | 27 000 | n.d. |

*not according to the invention
n.d. = not determinable

### Application Examples

Further typical preparations according to the invention are described below, but without limiting the invention to these examples.

Besides the preparation described here of the cosmetic preparations, the polymers A.1, A.2, A.3, A.4, A.5, A.6, A.7, A.8, A.9 or A.10 and also combinations thereof can be added to the resulting emulsion also after combining water phase and oil phase at 60-80° C. or to the cooled emulsion at about 40° C.

The invention also provides for the subsequent addition of the polyurethanes obtainable according to the invention to a cosmetic preparation in order to establish the desired viscosity.

The percentages are % by weight unless expressly described otherwise.

### O/W Emulsion

| Phase | Ingredient/INCI | F.3.1 | F.3.2 | F.3.3 | F.3.4 | F.3.5 |
|---|---|---|---|---|---|---|
| A | Aqua | ad 100 | ad 100 | ad 100 | ad 100 | ad 100 |
| | Glycerin | 3.0 | 5.50 | 4.50 | 5.00 | 3.5 |
| | Polymer A.1 | 3.0 | 1.5 | 0.8 | 2.0 | 2.5 |
| | Hydroxyethyl Acrylate/Sodium | | 1.0 | | 0.5 | |
| | Acryloyldimethyl Taurate Copolymer, | | | | | |
| | Squalane, Polysorbate 60 | | | | | |
| B | Glyceryl Stearate Citrate | 1.80 | 2.00 | 3.00 | 1.50 | 2 |
| | Sucrose Stearate | 1.00 | 1.20 | 2.00 | 2.20 | 1.5 |
| | Cetearyl Alcohol | 1.80 | 2.00 | 1.50 | 2.40 | 2.8 |
| | Ethylhexyl Palmitate | 6.00 | 5.00 | 3.50 | 3.00 | 5.5 |
| | Caprylic/Capric Triglyceride | 5.00 | 5.00 | 1.00 | 2.00 | 3.5 |
| | Octyldodecanol | 1.50 | 3.00 | 2.40 | 5.0 | 4.6 |
| | Dimethicone | 0.20 | 0.50 | 2.00 | 1.80 | 1.4 |
| C | Ammonium Acryloyldimethyltaurate/ | | | 0.5 | 0.1 | |
| | VP Copolymer | | | | | |
| | Carbomer | 0.05 | | | | 0.1 |
| D | Sodium Hydroxide | 0.02 | | | | 0.04 |
| E | Bisabolol | 0.5 | 0.3 | 0.20 | 0.35 | 1.0 |
| | Phenoxyethanol, Parabenmischung | 1.00 | 0.60 | 0.70 | 0.60 | 0.5 |
| | Parfum | 0.05 | 0.10 | 0.10 | 0.05 | 0.05 |

### Preparation:

Heat phases A and B separately to ca. 80° C. Stir phase C into phase B and then stir phase A into phase B/C and briefly homogenize.

Add phase D (if required) and cool to ca. 40° C. with stirring. Add components of phase E in succession to the emulsion and cool to room temperature with stirring. Briefly homogenize.

Instead of the O/W emulsion comprising polymer A.1, O/W emulsions comprising one or more of the polymers A.2, A.3, A.4, A.5, A.6, A.7, A.8, A.9 or A.10 are also prepared.

### Hydrodispersion

| Phase | Ingredient/INCI | F.4.1 | F.4.2 | F.4.3 | F.4.4 | F.4.5 |
|---|---|---|---|---|---|---|
| A | Stearyl Alcohol | 0.5 | 1.5 | | | 2.0 |
| | Cetyl Alcohol | | | 1.00 | 2.5 | |
| | C12-15 Alkyl Benzoate | | 2.5 | | 4.0 | |
| | Dicapryl Ether | | 4.0 | | | 6.0 |
| | Butylene Glycol Dicaprylate/Dicaprate | 4.0 | | 2.0 | 1.0 | |
| | Dicapryl Carbonate | | 2.0 | 3.0 | | 4.0 |
| | Cyclopentasiloxane, Cyclohexasiloxane | | | | 2.0 | 0.5 |
| | Simmondsia Chinensis | 2.0 | | 0.5 | | |
| | (Jojoba) Seed Oil | | | | | |
| | Shea Butter | | 2.0 | | 1.0 | |
| | Hydrogenated Polyisobutene | 3.0 | 1.0 | 7.0 | 0.5 | 2.0 |
| | Squalane | | | | 2.0 | 0.5 |
| | Vitamin E Acetate | 0.50 | | 0.25 | | 1.00 |
| B | Acrylate/C10-30 Alkyl | 0.3 | 0.1 | 0.2 | 0.15 | 0.2 |
| | Acrylat Crosspolymer | | | | | |
| C | Aqua | ad 100 | ad 100 | ad 100 | ad 100 | ad 100 |
| | Polyacrylamide, C13-14 | | 1.0 | 1.5 | 0.75 | |
| | Isoparaffin, Laureth-7 | | | | | |
| | Polymer A.1 | 2.5 | 2.0 | 0.9 | 1.5 | 3.0 |
| | Propylene Glycol | 3.00 | 5.0 | 2.5 | 7.50 | 10.0 |
| D | Sodium Hydroxide | 0.12 | 0.04 | 0.08 | 0.06 | 0.08 |
| E | Niacinamide | 0.30 | 3.0 | 1.5 | 0.5 | 0.20 |
| | Aqua | 2.0 | 10.0 | 5.0 | 2.0 | 2.0 |
| F | DMDM Hydantoin | | 0.60 | 0.45 | 0.25 | |
| | Methylparaben | 0.50 | | 0.25 | 0.15 | |
| | Phenoxyethanol | 0.50 | 0.40 | | 1.00 | |
| | Ethylhexylglycerin | | | 1.00 | | 0.80 |
| | Ethanol | 3.00 | 2.00 | 1.50 | | 7.00 |
| G | Fragrance | 0.20 | | 0.05 | | 0.40 |

### Preparation:

Heat phases A and C separately to ca. 80° C.

Stir phase B into phase A and then phase C into phase NB. Briefly homogenize. Add phase D and cool to ca. 40° C. with stirring. Add phase E and cool to ca. 30° C. with stirring. Add phase F and G to the emulsion and cool to room temperature with stirring. Briefly homogenize.

Instead of the hydrodispersion comprising polymer A.1, hydrodispersions comprising one or more of the polymers A.2, A.3, A.4, A.5, A.6, A.7, A.8, A.9 or A.10 are also prepared.

### Solids-Stabilized Emulsion

| Phase | Ingredient/INCI | F.5.1 | F.5.2 | F.5.3 | F.5.4 | F.5.5 |
|---|---|---|---|---|---|---|
| A | Mineral Oil | 4.0 | 6.0 | 16.0 | 10.0 | 6.0 |
| | Octyldodecanol | 9.0 | 9.0 | 5.0 | | |
| | Ethylhexyl Isononanoate | 9.0 | 9.0 | 6.0 | 5.0 | 8.0 |
| | Isohexadecane | 9.0 | 5.0 | | 4.0 | 8.0 |
| | Dimethicone | 0.5 | 2.0 | 1.0 | | 1.5 |
| | Cera Microcristallina, | | 0.35 | | 0.75 | 3.0 |
| | Paraffinum Liquidum | | | | | |
| | Phenyl trimethicone | 2.0 | | 1.0 | 2.5 | 3.0 |
| | Silica | 2.5 | | | 6.0 | 2.5 |
| | Aluminum Starch Octenylsuccinate | 2.0 | 1.0 | 0.5 | | |
| | Tapioca Starch | | 0.5 | | | |
| B | Titanium dioxide, coated | 1.0 | 0.5 | 3.0 | 2.0 | 4.0 |
| | Zinc oxide | 5.0 | 10.0 | 2.0 | 3.0 | |
| C | Ammonium Acryloyldimethyltaurate/ | 0.2 | | 1.0 | 0.5 | |
| | Beheneth-25 Methacrylate Crosspolymer | | | | | |
| D | Aqua | ad 100 | ad 100 | ad 100 | ad 100 | ad 100 |
| | Hydroxypropyl Methylcellulose | | | 0.1 | | 0.05 |
| | Sorbitol | 5.0 | 7.0 | 8.5 | 3.0 | 4.5 |
| | Polymer A.1 | 3.0 | 5.0 | 0.9 | 1.4 | 2.0 |
| E | Mixed parabens | 0.3 | 0.6 | 0.2 | | 0.4 |
| | Phenoxyethanol | 0.2 | 0.3 | 0.4 | 0.5 | 0.4 |
| | Diazolidinyl urea | | | 0.23 | 0.2 | |
| F | Fragrance | 0.2 | | 0.3 | 0.1 | |

### Preparation:

Heat phase A to 80° C. Add phase B to phase A and homogenize for 3 min. Stir in phase C.

Allow cellulose (if required) to preswell in water, then add the remaining ingredients of phase D and heat to 80° C.

Stir phase D into phase A+B+C and homogenize. Cool emulsion to ca. 40° C. with stirring and add phase E and F. Cool to room temperature (RT) with stirring and homogenize.

Instead of the solids-stabilized emulsion comprising polymer A.1, solids-stabilized emulsions comprising one or more of the polymers A.2, A.3, A.4, A.5, A.6, A.7, A.8, A.9 or A.10 are also prepared.

### Sunscreen Cream

| Phase | Ingredient/INCI | F.6.1 | F.6.2 | F.6.3 | F.6.4 | F.6.5 |
|---|---|---|---|---|---|---|
| A | Aqua | ad 100 | ad 100 | ad 100 | ad 100 | ad 100 |
| | Disodium EDTA | 0.1 | 0.1 | 0.1 | 0.1 | 0.1 |
| | Butylene Glycol | 3.00 | 7.50 | 8.0 | 7.50 | 5.00 |
| | Benzophenone-4 | 2.0 | | | 4.0 | |
| | Phenylbenzimidazole-sulfonic acid | | 0.50 | 4.00 | | 8.0 |
| | Triethanolamine | 1.0 | 0.25 | 2.0 | 2.0 | 4.0 |
| | Panthenol | 0.5 | | 0.75 | | 1.0 |
| | Polymer A.1 | 2.5 g | 0.5 g | 2.0 g | 4.0 | 1.5 |
| | Xanthan gum | | 0.3 | 0.15 | | 0.2 |
| B | Octocrylene | 8.0 | | | 7.5 | |
| | Ethylhexyl Methoxycinnamate, Diethylamino | 5.0 | 10.0 | 8.0 | 3.0 | 7.0 |
| | Hydroxybenzoyl Hexyl Benzoate | | | | | |
| | Steareth-21 | 2.0 | 3.0 | | 2.5 | |
| | Steareth-2 | 1.5 | | | | |
| | PEG-40 Stearate | | | 1.0 | | 2.0 |
| | Glycerin Monostearate SE | | 1.0 | 3.0 | 1.5 | 1.5 |
| | Dibutyl Adipate | 3.0 | 5.0 | 3.5 | 2.5 | 2.0 |
| | Cetearyl Alcohol | 2.0 | | | 0.5 | 3.0 |
| | Stearyl Alcohol | 1.5 | 3.0 | 2.5 | 0.6 | 2.0 |
| | Butyrospermum Parkii (Shea Butter) | 1.0 | 0.5 | | 1.0 | 1.5 |
| | Dimethicone | 1.0 | 0.5 | 1.5 | 0.8 | 2.0 |
| | PVP Hexadecene Copolymer | 0.20 | | 0.50 | 0.8 | 1.00 |
| | Bisabolol | 0.2 | 0.1 | | | 0.3 |
| C | DMDM Hydantoin | 0.5 | 0.5 | 0.5 | 0.5 | 0.75 |
| | Water, Aloe Barbadensis Leaf Juice | | | 0.5 | 1.0 | |
| | Tocopheryl Acetate | 0.60 | 0.5 | 0.4 | 0.25 | 0.3 |
| | Fragrance | 0.10 | 0.25 | 0.30 | 0.40 | 0.20 |

### Preparation:

Heat phases A and B separately to ca. 80° C.

Stir phase A into phase B and briefly homogenize.

Cool to ca. 40° C. with stirring. Add components of phase C in succession to the emulsion and cool to room temperature with stirring. Briefly homogenize.

Instead of the sunscreen cream comprising polymer A.1, sunscreen creams comprising one or more of the polymers A.2, A.3, A.4, A.5, A.6, A.7, A.8, A.9 or A.10 are also prepared.

### Silicone Emulsion

| Phase | Ingredient/INCI | F.7.1 | F.7.2 | F.7.3 | F.7.4 | F.7.5 |
|---|---|---|---|---|---|---|
| A | Water | ad 100 | ad 100 | ad 100 | ad 100 | ad 100 |
| | Butylene Glycol | 6.0 | 3.0 | 2.0 | 8.0 | 5.0 |
| | Polymer A.1 | 3.5 | 1.0 | 0.5 | 5.0 | 2.0 |
| | Xanthan Gum | | | 0.1 | | 0.15 |
| | Imidazolidinyl Urea | 0.3 | | 0.2 | | |
| B | Cetyl PEG/PPG-10/1 Dimethicone | 2.5 | 3.5 | 0.5 | 2.0 | 2.5 |
| | PEG-9 Dimethicone | 1.0 | 1.5 | 2.0 | 0.5 | |
| | PEG-14 Dimethicone | | 0.5 | 2.0 | | 2.5 |
| | PEG-11 Methyl Ether Dimethicone | 1.5 | 3.0 | 0.8 | 0.5 | |
| | Polyglyceryl-3 Disiloxane Dimethicone | 1.0 | 2.0 | 0.5 | | |
| | Cyclopentasiloxane, Caprylyl | | 0.5 | 5.0 | 2.5 | 3.5 |
| | Dimethicone Ethoxy Glucoside | | | | | |
| | Phenyl Trimethicone | 5.0 | 3.0 | 1.5 | 7.5 | |
| | Polymethylsilsesquioxane | 2.0 | 1.5 | 1.0 | 0.5 | |
| | Cyclopentasiloxane, Cyclohexasiloxane | 5.0 | 3.0 | 8.0 | | 10.0 |
| | Cetyl Dimethicone | 1.5 | 1.0 | 2.5 | 3.0 | 4.0 |
| | Paraben mixture | 0.2 | 0.2 | 0.2 | 0.2 | 0.2 |
| C | Sodium Citrate | 0.15 | 0.15 | 0.15 | 0.15 | 0.15 |
| | Monosodium Citrate | 0.05 | 0.05 | 0.05 | 0.05 | 0.05 |
| D | Bisabolol | 0.2 | 0.5 | 0.15 | 0.3 | 0.1 |
| | Fragrance | 0.1 | 0.05 | 0.05 | 0.1 | 0.15 |

### Preparation

Heat phases A and B separately to ca. 80° C.

Stir phase A into phase B and homogenize.

Stir phase C into phase A+B and homogenize.

Cool to ca. 40° C. with stirring. Add phase C and cool to 30° C. with stirring. Add phase D.

Cool to room temperature with stirring and briefly homogenize.

Instead of the silicone emulsion comprising polymer A.1, silicone emulsions comprising one or more of the polymers A.2, A.3, A.4, A.5, A.6, A.7, A.8, A.9 or A.10 are also prepared.

### Hydroxycarboxylic Acid Cream

| Phase | Ingredient/INCI | F.8.1 | F.8.2 | F.8.3 |
|---|---|---|---|---|
| A | Ceteareth-6, Stearyl Alcohol | 2.0 | | 2.5 |
| | Ceteareth-25 | 2.0 | | 2.5 |
| | PEG-100 Stearate, Glyceryl | | 3.5 | 0.5 |
| | Stearate | | | |
| | Polyglyceryl-3 Distearate | | 2.0 | |
| | Mineral Oil | 8.0 | 3.5 | 5.0 |
| | Cetearyl Ethylhexanoate | 7.0 | 5.5 | 4.0 |
| | Sorbitan Stearate | 0.5 | 1.5 | 0.5 |
| | Cera Alba | | 0.5 | 1.0 |
| | Cetyl Alcohol | 1.5 | 3.5 | 4.0 |
| | Dimethicone | 0.2 | 2.0 | 0.5 |
| B | Panthenol | 1.0 | 0.5 | 0.3 |
| | Propylene Glycol | 3.0 | 2.0 | 5.0 |
| | Polymer A.1 | 1.0 | 3.0 | 5.0 |
| | Hydroxy acid | 3.0 | 7.0 | 10.0 |
| | Aqua | ad 100 | ad 100 | ad 100 |
| C | Sodium Hydroxide | q.s. | q.s. | q.s. |
| D | Bisabolol | 0.2 | 0.1 | 0.3 |
| | Preservative | q.s. | q.s. | q.s. |
| | Fragrance | q.s. | q.s. | q.s. |

### Hint:

Alpha-hydroxy acids: for example lactic acid, citric acid, malic acid, glycolic acid

Dihydroxy acid: tartaric acid

Beta-hydroxy acid: salicylic acid

Adjust pH>3

### Preparation:

Heat phase A and B separately to ca. 80° C. Adjust pH of phase B to >3 using NaOH if necessary. Stir phase B into phase A, briefly homogenize.

Cool to ca. 40° C. with stirring, add components of phase D in succession, homogenize again.

Instead of the hydroxycarboxylic acid cream comprising polymer A.1,

hydroxycarboxylic acid creams comprising one or more of the polymers A.2, A.3, A.4, A.5, A.6, A.7, A.8, A.9 or A.10 are also prepared.

Emulsion with Deodorant Active Ingredient

| Phase | Ingredient/INCI | F.9.1 | F.9.2 | F.9.3 | F.9.4 | F.9.5 |
|---|---|---|---|---|---|---|
| | Ceteareth-6, Stearyl Alcohol | 1.5 | 2.0 | | | 1.0 |
| | Ceteareth-25 | 1.5 | 0.5 | | | 1.0 |
| | PEG-40 Hydrogenated Castor Oil | 0.5 | 1.0 | 2.0 | | |
| | Glyceryl Stearate | | 0.5 | 2.0 | 1.0 | |
| | Cetyl Alcohol | 2.0 | 1.0 | 0.5 | 2.5 | 0.2 |
| | Hydrogenated Coco-Glycerides | 2.0 | | | 1.0 | 0.5 |
| | Hydrogenated Polyisobutene | | 10.0 | 20.0 | 5.0 | 3.0 |
| | Decyl Oleate | 3.0 | 2.0 | | 8.0 | 5.0 |
| | Bis-PEG/PPG-14/14 Dimethicone, | 3.0 | 3.5 | 4.0 | 2.0 | 1.5 |
| | Cyclopentasiloxane | | | | | |
| | Talc | 3.0 | 2.5 | | | 1.5 |
| | Magnesium Aluminum Silicate | 1.0 | | 0.5 | 1.0 | 1.5 |
| B | Propylene Glycol | 10.0 | 5.0 | 7.5 | 20.0 | 15.0 |
| | Polymer A.1 | 0.5 | 1.0 | 3.0 | 3.5 | 2.0 |
| | Xanthan gum | 0.2 | 0.1 | | | 0.05 |
| | Cetyl Hydroxyethylcellulose | 0.3 | | | 0.1 | |
| | Aluminum Chlorohydrate | 5.0 | 10.0 | 20.0 | | |
| | Aluminum Zirconium | | 15.0 | | 50.0 | 20.0 |
| | Tetrachlorohydrex GLY | | | | | |
| | Aqua | ad 100 | ad 100 | ad 100 | ad 100 | ad 100 |
| C | Neutralizing agent | q.s. | q.s. | q.s. | q.s. | q.s. |
| D | Alcohol | 5.0 | 10.0 | 25.0 | 7.5 | 6.0 |
| | Allantoin | 0.1 | 0.1 | 0.1 | 0.1 | 0.1 |
| | Preservative | q.s. | q.s. | q.s. | q.s. | q.s. |
| | Fragrance | q.s. | q.s. | q.s. | q.s. | q.s. |

### Preparation

Heat phases A and B separately to ca. 80° C.

Stir phase B into phase A with homogenization. If necessary, use phase C to adjust to pH 4-5. Cool to ca. 40° C., add phase D and allow to cool to room temperature with stirring. Briefly homogenize.

Hint: adjust pH of the emulsion to 4-5

Instead of the emulsion with deodorant active ingredient comprising polymer A.1, emulsions with deodorant active ingredient comprising one or more of the polymers A.2, A.3, A.4, A.5, A.6, A.7, A.8, A.9 or A.10 are also prepared.

### Hair Removal Cream

| Phase | Ingredient/INCI | F.10.1 | F.10.2 | F.10.3 |
|---|---|---|---|---|
| A | Glyceryl Stearate | 1.0 | | |
| | Ceteareth-12 | | 1.0 | 2.0 |
| | Ceteareth-20 | | 1.0 | 2.0 |
| | Stearyl Alcohol | | 4.0 | 1.0 |
| | Cetyl Alcohol | 4.0 | | 1.0 |
| | Mineral Oil | | 6.0 | 4.0 |
| | Prunus Armeniaca | 3.0 | 1.0 | 2.0 |
| | (Apricot) Kernel Oil | | | |
| B | Propylene Glycol | 1.0 | 2.0 | 10.0 |
| | Calcium Carbonate | 10.0 | | |
| | Calcium Hydroxide | 7.0 | | |
| | Sodium Hydroxide | | 0.4 | 0.6 |
| | Calcium Thioglycolate | 5.0 | 3.0 | 5.0 |
| | Polymer A.1 | 3.0 | 1.5 | 2.0 |
| | Aqua | ad 100 | ad 100 | ad 100 |
| C | Tocopherol | 0.1 | 0.2 | 0.15 |
| | Bisabolol | 0.2 | 0.1 | 0.3 |
| | Fragrance | q.s. | q.s. | q.s. |

### Preparation

Heat phases A and B separately to ca. 80° C.

Stir phase B into phase A with homogenization, briefly homogenize.

Cool to ca. 40° C., add phase C, cool to RT with stirring and homogenize again.

Hint: Adjust pH of the emulsion to >10

Instead of the hair removal cream comprising polymer A.1, hair removal creams comprising one or more of the polymers A.2, A.3, A.4, A.5, A.6, A.7, A.8, A.9 or A.10 are also prepared.

### Conditioner Shampoo

| Ingredient/INCI | F.11.1 | F.11.2 | F.11.3 | F.11.4 |
|---|---|---|---|---|
| Aqua | ad 100 | ad 100 | ad 100 | ad 100 |
| Sodium Laureth Sulfate | 35.7 | | 30.0 | 12.0 |
| Cocamidopropyl Betaine | 13.5 | 15.0 | | |
| Disodium Cocoamphodiacetate | | 10.0 | | |
| Sodium Cocoamphoacetate | | | 6.0 | |
| Polysorbate 20 | | 5.0 | | |
| Decyl Glucoside | | 5.0 | | 1.5 |
| Laureth-3 | | 2.0 | | |
| Sodium Laureth Sulfate, Glycol | | | 3.0 | 2.0 |
| Distearate, Cocamide MEA, | | | | |
| Laureth-10 | | | | |
| Coco-Glucoside, Glyceryl Oleate | | | | 5.0 |
| Dimethicone | | | 2.0 | |
| Conditioning polymer | 2.0 | 0.5 | 0.75 | 0.4 |
| Polymer A.1 | 0.75 | 1.2 | 0.5 | 1.0 |
| PEG-150 Distearate | | 3.0 | | |
| Citric Acid | | q.s. | q.s. | |
| Preservative | q.s. | q.s. | q.s. | q.s. |
| Fragrance | q.s. | q.s. | q.s. | q.s. |
| Dye | q.s. | q.s. | q.s. | q.s. |
| Sodium Chloride | | | 1.0 | 1.0 |

Conditioning polymer is understood as meaning Polyquaternium-7, PQ-10, PQ-16, PQ-39, PQ-44, PQ-46, PQ-67, guar hydroxypropyltrimonium chloride, PQ-87, and combinations of these.

Instead of the conditioner shampoo comprising polymer A.1, conditioner shampoos comprising one or more of the polymers A.2, A.3, A.4, A.5, A.6, A.7, A.8, A.9 or A.10 are also prepared.

### Hair Conditioner

| Phase | Ingredient/INCI | F.12.1 | F.12.2 | F.12.3 | F.12.4 | F.12.5 |
|---|---|---|---|---|---|---|
| A | Water | ad 100 | ad 100 | ad 100 | ad 100 | ad 100 |
| | Polymer A.1 | 2.5 | 1.5 | 3.0 | 0.6 | 2.0 |
| | Hydroxyethylcellulose | 0.05 | 0.1 | | 0.2 | |
| | Propylene Glycol | 1.0 | 2.0 | | 0.8 | 0.5 |
| | Panthenol | 0.5 | | 0.75 | 0.25 | 0.3 |
| B | Quaternium-91, Cetearyl Alcohol, | 2.0 | | | | 1.5 |
| | Cetrimonium Methosulfate | | | | | |
| | Distearoylethyl Hydroxyethylmonium | | | 3.0 | 4.0 | |
| | Methosulfate, Cetearyl Alcohol | | | | | |
| | Hydrogenated Polyisobutene | 1.0 | 1.5 | | 1.0 | |
| | Cyclopentasiloxane | | | 2.0 | 1.0 | 0.5 |
| | Isopropyl Palmitate | | 1.0 | | 2.0 | |
| | Persea Gratissima (Avocado) Oil | | | | | 2.5 |
| | Steareth-2 | 0.75 | | 0.5 | | |
| | Ceteareth-6, Stearyl Alcohol | | 1.5 | | | 0.5 |
| | Ceteareth-25 | | 1.5 | | | |
| | Cetearyl Alcohol | 2.0 | 1.5 | 0.5 | 4.0 | |
| C | Acrylate/C10-30 alkyl acrylate copolymer | 0.1 | | | 0.2 | 0.15 |
| D | Cetrimonium Chloride | 1.5 | | 3.0 | | |
| | Conditioning Polymer | 2.0 | 6.0 | 3.0 | 1.5 | 0.8 |
| E | Preservative | q.s. | q.s. | q.s. | q.s. | q.s. |
| | Fragrance | q.s. | q.s. | q.s. | q.s. | q.s. |

Conditioning polymer is understood as meaning polyquaternium-7, PQ-10, PQ-16, PQ-39, PQ-44, PQ-46, PQ-67, guar hydroxypropyltrimonium chloride, PQ-87, and combinations of these.

### Preparation

Heat phases A and B separately to ca. 80° C.

Stir phase C into phase B, then stir phase A into phase B/C and briefly homogenize. Cool to ca. 50° C. with stirring, add components of phase D in succession and cool to ca. 30° C. with stirring. Add components of phase E in succession and cool to RT with stirring. Briefly homogenize.

Instead of the hair conditioner comprising polymer A.1, hair conditioners comprising one or more of the polymers A.2, A.3, A.4, A.5, A.6, A.7, A.8, A.9 or A.10 are also prepared.

1. A polymer P comprising, in polymerized-in form,
a) at least one polyisocyanate
b) at least one alcohol of the general formula I
R¹O—R²ₙOH (I)
where
R¹ is selected from C₆-C₄₀-alkyl, C₆-C₄₀-alkenyl, C₃-C₁₀-cycloalkyl, C₆-C₃₀-aryl and C₇-C₄₀-arylalkyl,
R² is selected from C₂-C₁₀-alkylene, C₆-C₁₀-arylene and C₇-C₁₀-arylalkylene,
n is selected from 0 to 200,
c) at least one hyperbranched polymer HB with functional groups, where, for the average number f of functional groups per molecule of the hyperbranched polymer, 3<f<100 applies,
with the proviso that the hyperbranched polymer is not selected from hyperbranched polyetherpolyols,
d) optionally at least one compound different from b) and c) and having a molecular weight of at least 300 g/mol comprising
i. at least two OH groups and
ii. at least two groups selected from ether groups and ester groups,
e) optionally further compounds different from b) to d) and having 1 to 10 groups that are reactive toward isocyanate groups per molecule.

2. The polymer P according to claim 1, wherein the hyperbranched polymer HB is selected from the group consisting of hyperbranched polyureas, polycarbonates, polyesters, polyester carbonates, polyether carbonates, polyether esters, polyether ester carbonates, polyurethanes, polyisocyanurates, polyamides, polyamines, polyurethaneureas, polyester amides, polyester amines, and polyether amines.

3. A process for preparing polymer P according to claim 1 comprising polymerizing components a) to e).

4. The process according to claim 3, wherein the hyperbranched polymer HB is a hyperbranched polycarbonate that is obtainable by:
i. preparing a condensation product K by reacting an organic carbonate or a phosgene derivative with an alcohol comprising at least three OH groups, and subsequently
ii. converting the condensation product K to the hyperbranched polycarbonate, where the quantitative ratio of the OH groups to the carbonate or phosgene groups is selected such that the condensation product K has, on average, either
(1) one carbonate or carbamoyl chloride group and more than OH group, or
(2) one OH group and more than one carbonate or carbamoyl group.

5. The process according to claim 3, wherein in the range from 5 to 95 mol % of the functional groups of the hyperbranched polymer HB present before the polymerization are consumed by the polymerization.

6. A polymer P which is obtainable by the process according to claim 4.

7. The polymer according to claim 1, wherein a condensation product K forms the basis of the hyperbranched polymer HB and wherein this condensation product K comprises, in condensed-in form, at least one polyetherol that is obtainable by alkoxylation of at least trifunctional alcohols with C₂-C₄-alkylene oxide.

8. The polymer P according to claim 1, wherein the hyperbranched polymer HB has a number-average molecular weight Mₙ of at least 300 g/mol.

9. The polymer P according to claim 1, wherein b) comprises a C₁₂-C₃₀-alcohol that has been ethoxylated with 3 to 100 mol of ethylene oxide per mole of alcohol.

10. The polymer P according to claim 1, wherein d) comprises a polyetherdiol with a number-average molecular weight Mₙ in the range from 1500 to 12 000 g/mol.

11. A modified polymer MP1 obtainable by reacting at least some of the functional groups of a polymer P according to claim 1 with compounds that are reactive toward these functional groups.

12. The modified polymer MP1 according to claim 11, wherein the compounds that are reactive toward the functional groups of the polymer P comprise isocyanate groups.

13. A modified polymer MP2 obtainable by reacting the modified polymer MP1 according to claim 11 with a compound such that MP2, after the reaction of MP1, comprises structures which are selected from carboxylate, sulfonate, diol, sugars, polar polymer chains, nonpolar PIB chains, silicone chains and amphiphilic surfactant chains.

14. A method of making aqueous preparations, the method comprising: obtaining the polymer P according to claim 1, mixing the polymer P with aqueous cosmetic ingredients, wherein the polymer P is effective as thickener.

15. A cosmetic preparation comprising at least one polymer P according to claim 1, or a modified polymer MP1 obtainable by reacting at least some of the functional groups of the polymer P according to claim 1 with compounds that are reactive toward these functional groups, or a modified polymer MP2 obtainable by reacting MP1 with a compound such that MP2, after the reaction of MP1, comprises structures which are selected from carboxylate, sulfonate, diol, sugars, polar polymer chains, nonpolar PIB chains, silicone chains and amphiphilic surfactant chains.

16. The method of claim 14 further comprising: after obtaining the polymer P according to claim 1, reacting some of the functional groups of the polymer P with compounds that are reactive toward these functional groups to form a modified polymer MP1, and mixing polymer MP1 with aqueous cosmetic ingredients, wherein the polymer MP1 is effective as thickener.

17. The method of claim 16 further comprising: after obtaining the polymer MP1, a compound such that MP2, after the reaction of MP1, comprises structures which are selected from carboxylate, sulfonate, diol, sugars, polar polymer chains, nonpolar PIB chains, silicone chains and amphiphilic surfactant chains, and mixing polymer MP2 with aqueous cosmetic ingredients, wherein the polymer MP2 is effective as thickener.

18. The polymer P according to claim 1, wherein for the average number f of functional groups per molecule of the hyperbranched polymer 3<f<20.

19. The polymer P according to claim 2, wherein the hyperbranched polymer HB is selected from hyperbranched polyureas, polyurethanes, polycarbonates, polyether carbonates, polyesters and polyether amines.

20. The process according to claim 5, wherein in the range from 50 to 90 mol % of the functional groups of the hyperbranched polymer HB present before the polymerization are consumed by the polymerization.
